(19) United States
(12) Patent Application Publication
(10) Pub. No.: US 2007/0238793 A1
(43) Pub. Date: Oct. 11, 2007

(54) Structural carotenoid analogs or derivatives for the modulation of systemic and/or target organ redox status

(76) Inventor: Samuel F. Lockwood, Lago Vista, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/636,401

(22) Filed: Dec. 7, 2006

Related U.S. Application Data
(60) Provisional application No. 60/748,385, filed on Dec. 7, 2005.

(51) Int. Cl. A61K 31/01

(52) U.S. Cl. 514/762

(57) ABSTRACT

Methods for the reduction or prevention of oxidative stress in a human subject comprising administering to the human subject an effective amount of a composition comprising xanthophyll carotenoids, or analogs or derivatives of astaxanthin, lutein, zeaxanthin, lycoxanthin, lycophyll, or lycopene are described. Also described are compositions comprising xanthophyll carotenoids, or analogs or derivatives of astaxanthin, lutein, zeaxanthin, lycoxanthin, lycophyll, or lycopene, the compositions being effective for the reduction or prevention of oxidative stress in a human subject.

### PRIORITY CLAIM

The present application claims the benefit of priority under 35 U.S.C. §119 to U.S. Provisional Application No. 60/748,385, filed Dec. 7, 2005. The prior applications are considered part of the present application, and the contents thereof are hereby incorporated by reference in their entirety as though fully set forth herein.

### BACKGROUND OF THE INVENTION

1. Field of the Invention

The present invention generally relates to the fields of medicinal and synthetic chemistry. Specifically, the invention relates to the use of carotenoids, and in particular xanthophyll carotenoids, including analogs, derivatives, and intermediates thereof, as therapeutic agents that that modulate whole body and target organ redox status.

2. Description of the Related Art

Aerobic organisms use oxidative processes, such as oxidative catabolism, metabolism, and phosphorylation, to extract energy from food molecules. These processes provide the organisms with a defense against attacks due to infections or other chemical disturbances. These processes also produce free radicals, such as reactive oxygen species (“ROS”) and reactive nitrogen species.

Oxidative stress, an undesirable imbalance where oxidants outnumber antioxidants, can arise if the rate of ROS production overwhelms existing antioxidant defenses. In such circumstances, a series of cellular responses can occur that can lead to an even greater increase in ROS production. Excessive ROS production, and its otherwise ineffective regulation can be detrimental to cells and tissue, potentially causing cellular damage that ultimately can lead to cell death (apoptosis). Oxidative stress-associated damage also can cause undesirable changes to the structural and functional integrities of cells that can lead to the propagation of cells instead of apoptosis. Additionally, cellular macromolecules that have undergone oxidative damage can initiate immune responses that can result in disease (see generally, D. G. Lindsay et al. (2002) Mol. Aspects. of Med. 23:1-38). While oxidative stress may not be responsible for initiating or otherwise initiating disease, the severity of certain diseases can be influenced by any resultant oxidative stress. ROS can result from viral and/or bacterial infections, and can be produced by exposure to environmental oxidants, toxicants, and heavy metals, which can disturb the equilibrium between cellular reduction and oxidative reactions and otherwise disrupt normal biological functions.

Optimal control of ROS levels is important for cellular homeostasis, for example. In physiologic concentrations, certain ROS are important mediators and components of a number of signal transduction cascades. When present at excessive concentrations, however, ROS can damage cellular biomolecules (e.g., proteins, lipids, and nucleic acids). ROS have been implicated in the acceleration of cellular senescence, neurodegeneration, malignancy, and atherosclerosis, among other pathologies. Consequently, it has been theorized that therapeutic modulation of ROS levels may prevent, delay the onset of, or even ameliorate these conditions.

Oxidation of DNA can produce a number of molecular alterations, including, for example, cleavage, cross-linkage between DNA and proteins, and oxidation of purines. While cells repair many of the DNA mutations caused by oxidative damage using several innate DNA repair pathways, over time deleterious genetic mutations nonetheless accumulate, resulting ultimately in carcinogenesis, and senescence. Mutations resulting from oxidative stress are also believed to influence the pathogenesis of several neurological and age-associated diseases, such as, for example, atherosclerosis, autoimmune disease, cancer, cardiovascular disease, cataracts, dementia, diabetes and diabetic vasculopathy, and neurodegenerative diseases, to name but a few. A tightly regulated network of intracellular mechanisms has therefore evolved to protect and ensure genomic stability, and to address oxidative stress. Among the more prominent intracellular mechanisms responsible for modulating oxidative stress are the “thioredoxin system” and the antioxidant enzymes catalase, glutathione peroxidase and superoxide dismutase.

In addition to the intracellular mechanisms mentioned above, oxidative stress can be modulated by exogenous ligands that activate the cyclic AMP (cAMP)-dependent protein kinase (PKA). Elevated [cAMP]ᵢ blocks many biological effects of hydrogen peroxide (H₂O₂), including filamin redistribution and increased permeability in endothelial cells, P-glycoprotein downregulation in prostate cancer cells, neutrophil adherence to human umbilical vein endothelial cells (HUVEC), and c-Jun N-terminal kinase activation in Chinese hamster V79 cells. Additionally, extracellular adenosine inhibits oxidative burst in neutrophils and protects against ischemia-reperfusion renal injury through A2a-mediated [cAMP]ᵢ increase.

In additions to compounds that stimulate the aforementioned signaling pathways, oxidative stress may be modulated by certain low molecular weight antioxidants such as selenium and certain phytochemicals, such as ascorbic acid (vitamin C), α-tocopherol (vitamin E), β-carotene, and derivatives thereof, as well as certain plant-derived antioxidants and food supplements. For example, β-carotene is capable of quenching singlet oxygen and has been shown to exert antioxidant effects in vitro (K. Fukuzawa et al. (1998) Biofactors 7:31-40). The isoprenoid ubiquinone (coenzyme Q10) is an electron carrier in the inner mitochondrial membrane. In its reduced form, ubiquinone has been shown to protect lipids, proteins, and DNA against oxidative damage (H. Nohl et al. (1998) Ann. NY Acad. Sci. 854:394-409). Polyisoprenyl diphosphates, which also are isoprenoids, exert antioxidant effects and, at nanomolar concentrations, inhibit phospholipase D and superoxide generation in human neutrophils. See B. Levy et al. (1997) Nature 389:985-990; B. Levy et al (2002) Cell. Mol Life Sci. 59:729-741. Polyisoprenyl monophosphates, however, do not exert a similar antioxidant effect at equimolar concentrations. Yet another class of isoprenoids, prenylated proteins, and specifically the heterotrimeric G proteins and the small GTP-binding proteins (i.e., Ras, Rac, and Rap), whose proper membrane localization and activation state are dependent upon isoprenylation, have been shown to function as ROS and RNS regulators. See M. Santillo et al. (1996) Biochem. Biophysic. Res. Comm. 229:739-745; M. Sundaresan et al. (1996) Biochem. J. 318:379-382; H. Chen et al. (2000) Hypertension 36:923-928.

The clinical effectiveness of these compounds, however, is a matter of on-going debate due in-part to the dual antioxidant and pro-oxidant effects of many of these agents, such as ascorbic acid and β-carotene, for example, and also due in-part to high minimum inhibitory concentrations (IC₅₀ values) necessary for producing a meaningful antioxidant effect. For example, each of the three chemically-characterized antioxidants (i.e., ascorbic acid, α-tocopherol, and β-carotene) and derivatives thereof requires high concentrations in the nanomolar to micromolar-range to exert any meaningful antioxidant effect. Such high concentrations, of course, implicate toxicity issues. Thus, while, many of these antioxidants are believed to decrease mutation rates, the therapeutic utility of each remains highly uncertain.

In view of the foregoing, it would be desirable to find an agent capable of preventing, delaying the onset of, or even ameliorating oxidative DNA damage, cellular senescence, neurodegeneration, malignancy, and atherosclerosis, and other pathologies in a manner heretofore unattainable with conventional antioxidant agents. Furthermore, it would be desirable to find an agent capable of treating, preventing, delaying the onset of, and/or otherwise ameliorating the symptoms of oxidative stress-associated diseases.

Antioxidant Properties of Carotenoids

Free radicals are highly reactive molecules having one or more unpaired electrons in their outer orbital. Free radicals are involved in normal metabolism, and are always present in the human body, but normally at very low concentrations. There is considerable interest in understanding free radical biochemistry, since changes in the bioavailability of these molecules are believed to be involved in the early stages and progression of several diseases, such as cancer, inflammatory disease and cardiovascular, among others.

Carotenoids are a group of natural pigments produced principally by plants, yeast, and microalgae. The family of related compounds now numbers greater than 750 described members, exclusive of Z and E isomers. Humans and other animals cannot synthesize carotenoids de novo and must obtain them from their diet. All carotenoids share common chemical features, such as a polyisoprenoid structure, a long polyene chain forming the chromophore, and near symmetry around the central double bond. Tail-to-tail linkage of two C₂₀ geranyl-geranyl diphosphate molecules produces the parent C₄₀ carbon skeleton. Carotenoids without oxygenated functional groups are called “carotenes”, reflecting their hydrocarbon nature; oxygenated carotenes are known as “xanthophylls.” “Parent” carotenoids may generally refer to those natural compounds utilized as starting scaffold for structural carotenoid analog synthesis. Carotenoid derivatives may be derived from a naturally occurring carotenoid. Naturally occurring carotenoids may include lycopene, lycophyll, lycoxanthin, astaxanthin, beta-carotene, lutein, zeaxanthin, and/or canthaxanthin to name a few.

Cyclization at one or both ends of the molecule yields 7 identified end groups (illustrative structures shown in FIG. 1). Examples of uses of carotenoid derivatives and analogs are illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and PCT International Application Number PCT/US2003/023706 filed on Jul. 29, 2003, entitled “STRUCTURAL CAROTENOID ANALOGS FOR THE INHIBITION AND AMELIORATION OF DISEASE” by Lockwood et al. (International Publication Number WO 2004/011423 A2, published on Feb. 5, 2004) both of which are incorporated by reference as though fully set forth herein.

Documented carotenoid functions in nature include light-harvesting, photoprotection, and protective and sex-related coloration in microscopic organisms, mammals, and birds, respectively. A relatively recent observation has been the protective role of carotenoids against age-related diseases in humans as part of a complex antioxidant network within cells. This role is dictated by the close relationship between the physicochemical properties of individual carotenoids and their in vivo functions in organisms. The long system of alternating double and single bonds in the central part of the molecule (delocalizing the π-orbital electrons over the entire length of the polyene chain) confers the distinctive molecular shape, chemical reactivity, and light-absorbing properties of carotenoids. Additionally, isomerism around C═C double bonds yields distinctly different molecular structures that may be isolated as separate compounds (known as Z (“cis”) and E (“trans”), or geometric, isomers). Of the more than 750 described carotenoids, an even greater number of the theoretically possible mono-Z and poly-Z isomers are sometimes encountered in nature. The presence of a Z double bond creates greater steric hindrance between nearby hydrogen atoms and/or methyl groups, so that Z isomers are generally less stable thermodynamically, and more chemically reactive, than the corresponding all-E form. The all-E configuration is an extended, linear, and rigid molecule. Z-isomers are, by contrast, not simple, linear molecules (the so-called “bent-chain” isomers). The presence of any Z in the polyene chain creates a bent-chain molecule. The tendency of Z-isomers to crystallize or aggregate is much less than the all-E isomers. Additionally, Z isomers are more readily solubilized, absorbed, and transported in vivo than their all-E counterparts. This has important implications for enteral (e.g., oral) and parenteral (e.g., intravenous, intra-arterial, intramuscular, and subcutaneous) dosing in mammals.

Carotenoids with chiral centers may exist either as the R (rectus) or S (sinister) configurations. As an example, astaxanthin (with 2 chiral centers at the 3 and 3′ carbons) may exist as 4 possible stereoisomers: 3S, 3′S; 3R, 3′S and 3S, 3′R (identical meso forms); or 3R, 3′R. The relative proportions of each of the stereoisomers may vary by natural source. For example, Haematococcus pluvialis microalgal meal is 99% 3S, 3′S astaxanthin, and is likely the predominant human evolutionary source of astaxanthin. Krill (3R,3′R) and yeast sources yield different stereoisomer compositions than the microalgal source. Synthetic astaxanthin, produced by large manufacturers such as Hoffmann-LaRoche AG, Buckton Scott (USA), or BASF AG, are provided as defined geometric isomer mixtures of a 1:2:1 stereoisomer mixture [3S, 3′S; 3R, 3′S, 3′R,3S (meso); 3R, 3′R] of non-esterified, free astaxanthin. Natural source astaxanthin from salmon fish is predominantly a single stereoisomer (3S,3′S), but does contain a mixture of geometric isomers. Astaxanthin from the natural source Haematococcus pluvialis may contain nearly 50% Z isomers. As stated above, the Z conformational change may lead to a higher steric interference between the two parts of the carotenoid molecule, rendering it less stable, more reactive, and more susceptible to reactivity at low oxygen tensions. In such a situation, in relation to the all-E form, the Z forms: (1) may be degraded first; (2) may better suppress the attack of cells by reactive oxygen species such as superoxide anion; and (3) may preferentially slow the formation of radicals. Overall, the Z forms may initially be thermodynamically favored to protect the lipophilic portions of the cell and the cell membrane from destruction. It is important to note, however, that the all-E form of astaxanthin, unlike β-carotene, retains significant oral bioavailability as well as antioxidant capacity in the form of its dihydroxy- and diketo-substitutions on the β-ionone rings, and has been demonstrated to have increased efficacy over β-carotene in most studies. The all-E form of astaxanthin has also been postulated to have the most membrane-stabilizing effect on cells in vivo. Therefore, it is likely that the all-E form of astaxanthin in natural and synthetic mixtures of stereoisomers is also extremely important in antioxidant mechanisms, and may be the form most suitable for particular pharmaceutical preparations.

The antioxidant mechanism(s) of carotenoids, and in particular astaxanthin, includes singlet oxygen quenching, direct radical scavenging, and lipid peroxidation chain-breaking. The polyene chain of the carotenoid absorbs the excited energy of singlet oxygen, effectively stabilizing the energy transfer by delocalization along the chain, and dissipates the energy to the local environment as heat. Transfer of energy from triplet-state chlorophyll (in plants) or other porphyrins and proto-porphyrins (in mammals) to carotenoids occurs much more readily than the alternative energy transfer to oxygen to form the highly reactive and destructive singlet oxygen (¹O₂). Carotenoids may also accept the excitation energy from singlet oxygen if any should be formed in situ, and again dissipate the energy as heat to the local environment. This singlet oxygen quenching ability has significant implications in cardiac ischemia, macular degeneration, porphyria, and other disease states in which production of singlet oxygen has damaging effects. In the physical quenching mechanism, the carotenoid molecule may be regenerated (most frequently), or be lost. Carotenoids are also excellent chain-breaking antioxidants, a mechanism important in inhibiting the peroxidation of lipids. Astaxanthin can donate a hydrogen (H.) to the unstable polyunsaturated fatty acid (PUFA) radical, stopping the chain reaction. Peroxyl radicals may also, by addition to the polyene chain of carotenoids, be the proximate cause for lipid peroxide chain termination. The appropriate dose of astaxanthin has been shown to completely suppress the peroxyl radical chain reaction in liposome systems. Astaxanthin shares with vitamin E this dual antioxidant defense system of singlet oxygen quenching and direct radical scavenging, and in most instances (and particularly at low oxygen tension in vivo) is superior to vitamin E as a radical scavenger and physical quencher of singlet oxygen.

Carotenoids, and in particular astaxanthin, are potent direct radical scavengers and singlet oxygen quenchers and possess all the desirable qualities of such therapeutic agents for inhibition or amelioration of ischemia-reperfusion (I/R) injury. Synthesis of novel carotenoid derivatives with “soft-drug” properties (i.e. activity in the derivatized form), with physiologically relevant, cleavable linkages to pro-moieties, can generate significant levels of free carotenoids in both plasma and solid organs. This is critically important, for in mammals, diesters of carotenoids generate the non-esterified or “free” parent carotenoid, and may be viewed as elegant synthetic and novel delivery vehicles with improved properties for delivery of free carotenoid to the systemic circulation and ultimately to target tissue. In the case of non-esterified, free astaxanthin, this is a particularly useful embodiment (characteristics specific to non-esterified, free astaxanthin below):
Lipid soluble in natural form; may be modified to become more water soluble;
Molecular weight of 597 Daltons [size <600 Daltons (Da) readily crosses the blood brain barrier, or BBB];
Long polyene chain characteristic of carotenoids effective in singlet oxygen quenching and lipid peroxidation chain breaking;
No pro-vitamin A activity in mammals (eliminating concerns of hypervitaminosis A and retinoid toxicity in humans).

The administration of antioxidants, which are potent singlet oxygen quenchers and direct radical scavengers, particularly of superoxide anion, should limit hepatic fibrosis and the progression to cirrhosis by affecting the activation of hepatic stellate cells early in the fibrogenetic pathway. Reduction in the level of ROS by the administration of a potent antioxidant can therefore be crucial in the prevention of the activation of both HSC and Kupffer cells. This protective antioxidant effect appears to be spread across the range of potential therapeutic antioxidants, including water-soluble (e.g., vitamin C, glutathione, resveratrol) and lipophilic (e.g., vitamin E, β-carotene, astaxanthin) agents. Therefore, a co-antioxidant derivative strategy in which water-soluble and lipophilic agents are combined synthetically is a particularly useful embodiment.

Vitamin E is generally considered the reference antioxidant. When compared with vitamin E, carotenoids are more efficient in quenching singlet oxygen in homogeneous organic solvents and in liposome systems. They are better chain-breaking antioxidants as well in liposomal systems. They have demonstrated increased efficacy and potency in vivo. They are particularly effective at low oxygen tension, and in low concentration, making them extremely effective agents in disease conditions in which ischemia is an important part of the tissue injury and pathology. These carotenoids also have a natural tropism for the liver after oral administration. Therefore, therapeutic administration of carotenoids should provide a greater benefit in limiting fibrosis than vitamin E.

Problems related to the use of some carotenoids and structural carotenoid analogs include: (1) the complex isomeric mixtures, including non-carotenoid contaminants, provided in natural and synthetic sources leading to costly increases in safety and efficacy tests required by such agencies as the FDA; (2) limited bioavailability upon administration to a human subject; and (3) the differential induction of cytochrome P450 enzymes (this family of enzymes exhibits species-specific differences which must be taken into account when extrapolating animal work to human studies).

### SUMMARY OF THE INVENTION

Provided for herein are pharmaceutical compositions and methods of their use that are suited to inhibiting, reducing or ameliorating systemic and/or target organ oxidative stress in a human subject who would benefit from reduced oxidant levels.

In one embodiment, a method of inhibiting, reducing or ameliorating oxidative stress systemically and/or in a body organ of a human subject may include administering to the human subject an effective amount of a pharmaceutically acceptable formulation including a xanthophyll or other carotenoid or a synthetic analog or derivative thereof. In an embodiment, the formulation may include astaxanthin, lutein and/or zeaxanthin. Inhibiting, reducing or ameliorating systemic or target organ oxidative stress may reduce at least some of the pathological consequences associated with elevated systemic and/or target organ oxidative stress, such as, for example, chronic inflammation, increased risk of breast or prostate cancer, cardiovascular disease (e.g., hypertension, atherosclerosis), obesity, chronic obstructive pulmonary disease (COPD), certain neurodegenerative conditions (e.g., Parkinson's disease), chronic fatigue syndrome, Systemic Lupus Erythematosis (SLE), and cognitive decline.

In an embodiment, a method of inhibiting, reducing or ameliorating oxidative stress in the heart of a human subject may include administering to the human subject an effective amount of a pharmaceutically acceptable formulation including a xanthophyll or other carotenoid or a synthetic analog or derivative thereof. In an embodiment, the formulation may include astaxanthin, lutein and/or zeaxanthin. Inhibiting, reducing or ameliorating cardiac oxidative stress may lessen the severity of certain pathophysiological conditions associated with elevated oxidative stress in the heart, such as, for example, chronic heart failure, cardiac hypertrophy, myocardial infarction, cardiac inflammation and contractile dysfunction.

In an embodiment, a method of modulating the redox status of the body or of a body organ of a human subject may include administering to the human subject an effective amount of a pharmaceutically acceptable formulation including a xanthophyll or other carotenoid or a synthetic analog or derivative thereof. In an embodiment, the formulation may include astaxanthin, lutein and/or zeaxanthin.

In some embodiments, carotenoid analogs or carotenoid derivatives suited for use in the embodiments described herein include those derivatives or analogs that undergo chemical and/or enzymatic breakdown in a human subject's body, in the digestive tract, in the serum, in the plasma, or in a cell, wherein at least one of the breakdown products is astaxanthin, or a derivative or an analog of astaxanthin.

In some embodiments, the administration of carotenoids, xanthophyll carotenoids or structural analogs or derivatives of carotenoids by one skilled in the art—including consideration of the pharmacokinetics and pharmacodynamics of therapeutic drug delivery—is expected to inhibit and/or ameliorate disease conditions associated with administering xanthophyll carotenoid or a synthetic analog or derivative thereof to a human subject, including but not limited to the production of oxidized lipids, and LDL. In some of the foregoing embodiments, analogs or derivatives of carotenoids administered to cells may be at least partially water-soluble.

“Water-soluble” structural carotenoid analogs or derivatives are those analogs or derivatives that may be formulated in aqueous solution, either alone or with one or more excipients. Water-soluble carotenoid analogs or derivatives may include those compounds and synthetic derivatives that form molecular self-assemblies, and may be more properly termed “water dispersible” carotenoid analogs or derivatives. Water-soluble and/or “water-dispersible” carotenoid analogs or derivatives may be preferred in some embodiments.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/ml-10 mg/mL. In certain embodiments, water-soluble carotenoid analogs or derivatives may have a water solubility of greater than about 20 mg/mL. In certain embodiments, water-soluble carotenoid analogs or derivatives may have a water solubility of greater than about 25 mg/mL. In some embodiments, water-soluble carotenoid analogs or derivatives may have a water solubility of greater than about 50 mg/mL.

In some embodiments, water-soluble analogs or derivatives of carotenoids may be administered to a human subject alone or in combination with additional xanthophyll carotenoids or structural analogs or derivatives. In some embodiments, water-soluble analogs or derivatives of carotenoids may be administered to a human subject alone or in combination with other antioxidants.

In some embodiments, methods to modulate the redox status of the body or of a body organ, or to inhibit or reduce systemic or target organ oxidative stress may include administering to the human subject an effective amount of a pharmaceutically acceptable formulation including a carotenoid. In some embodiments, a carotenoid may have the structure:

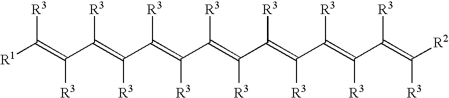

where each R³ is independently hydrogen or methyl, and where each R¹ and R² is independently: 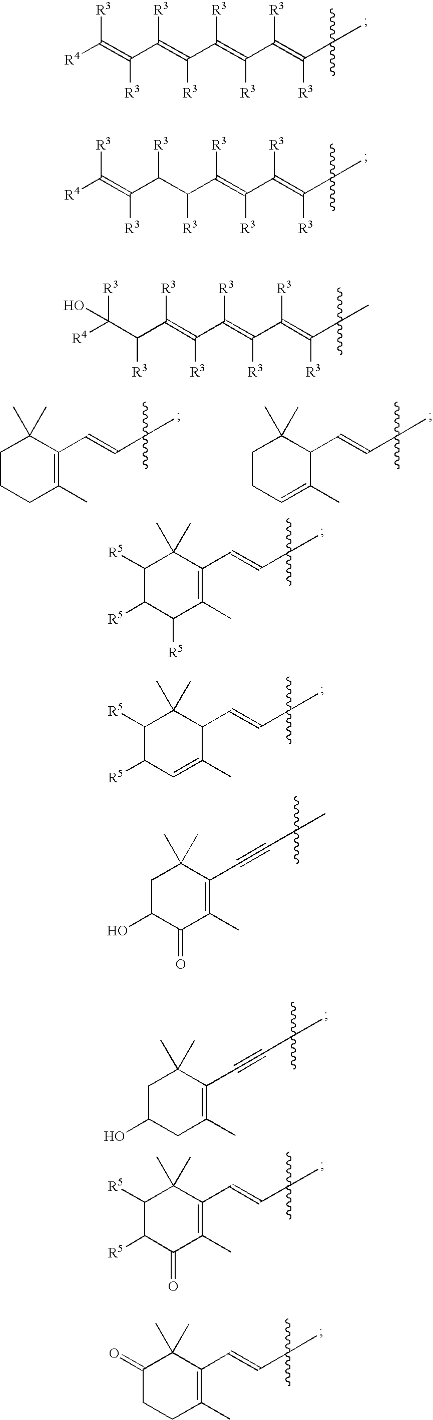 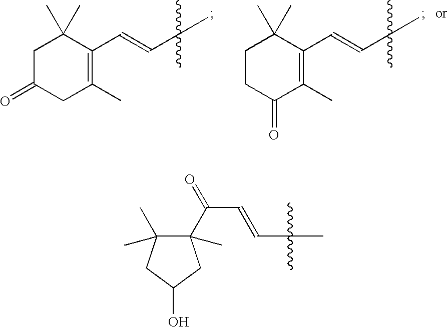
where R⁴ is hydrogen, methyl, or —CH₂OH; and where each R⁵ is independently hydrogen or —OH.

In some embodiments, methods to modulate the redox status of the body or of a body organ, or to inhibit or reduce systemic or target organ oxidative stress may include administering to the human subject an effective amount of a pharmaceutically acceptable formulation including a synthetic analog or derivative of a carotenoid. The synthetic analog or derivative of the carotenoid may have the structure

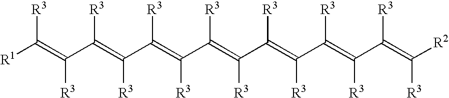

where each R³ is independently hydrogen or methyl, and where each R¹ and R² is independently: 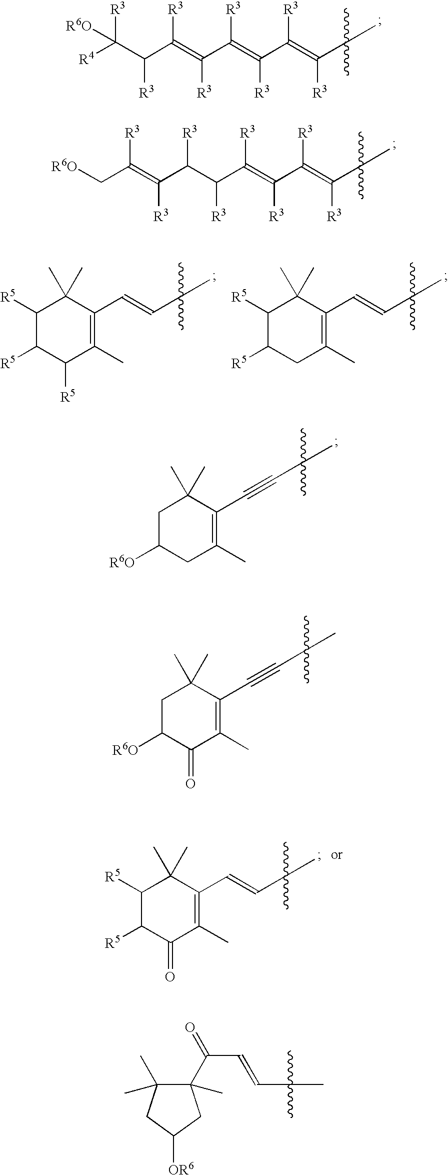
where R⁴ is hydrogen or methyl; where each R⁵ is independently hydrogen, —OH, or —OR⁶ wherein at least one R⁵ group is —OR⁶; wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂), —CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to modulate the redox status of the body or of a body organ, or to inhibit or reduce systemic or target organ oxidative stress of the human subject.

Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids include, but are not limited to, quercetin, xanthohumol, isoxanthohumol, or genistein. Selection of the co-antioxidant should not be seen as limiting for the therapeutic application of the current invention.

Embodiments directed to pharmaceutical compositions may further include appropriate vehicles for delivery of said pharmaceutical composition to a desired site of action (i.e., the site a human subject's body where the biological effect of the pharmaceutical composition is most desired). Pharmaceutical compositions including xanthophyll carotenoids or analogs or derivatives of astaxanthin, lutein or zeaxanthin that may be administered orally or intravenously may be particularly advantageous for and suited to embodiments described herein. In yet a further embodiment, an injectable astaxanthin formulation or a structural analog or derivative may be administered with a astaxanthin, zeaxanthin or lutein structural analog or derivative and/or other carotenoid structural analogs or derivatives, or in formulation with antioxidants and/or excipients that further the intended purpose. In some embodiments, one or more of the xanthophyll carotenoids or synthetic analogs or derivatives thereof may be at least partially water-soluble.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings.

FIG. 1 depicts a graphic representation of several examples of the structures of several xanthophyll carotenoids and synthetic derivatives or analogs that may be used according to some embodiments.

FIG. 2 depicts a time series of the UV/Vis absorption spectra of the disodium disuccinate derivative of natural source lutein in water.

FIG. 3 depicts a UV/Vis absorption spectra of the disodium disuccinate derivative of natural source lutein in water (λₘₐₓ=443 nm), ethanol (λₘₐₓ=446 nm), and DMSO (λₘₐₓ=461 nm).

FIG. 4 depicts a UV/Vis absorption spectra of the disodium disuccinate derivative of natural source lutein in water (λₘₐₓ=442 nm) with increasing concentrations of ethanol.

FIG. 5 depicts a time series of the UV/Vis absorption spectra of the disodium diphosphate derivative of natural source lutein in water.

FIG. 6 depicts a UV/Vis absorption spectra of the disodium diphosphate derivative of natural source lutein in 95% ethanol (λₘₐₓ=446 nm), 95% DMSO (λₘₐₓ=459 nm), and water (λₘₐₓ=428 nm).

FIG. 7 depicts a UV/Vis absorption spectra of the disodium diphosphate derivative of natural source lutein in water (λₘₐₓ=428 nm) with increasing concentrations of ethanol.

FIG. 8 depicts a mean percent inhibition (±SEM) of superoxide anion signal as detected by DEPMPO spin-trap by the disodium disuccinate derivative of natural source lutein (tested in water).

FIG. 9 depicts a mean percent inhibition (±SEM) of superoxide anion signal as detected by DEPMPO spin-trap by the disodium diphosphate derivative of natural source lutein (tested in water).

FIG. 10 depicts 3 stereoisomers comprising the statistical mixture of stereoisomers of the disodium disuccinate diester of astaxanthin used in Examples 1-5.

FIG. 12 is a representative depiction of a series whole-body time-course PEDRI images of TEMPONE distribution in a mouse treated with disodium disuccinate diester of astaxanthin (10 mg/kg I.V. for 4 days prior to imaging) according to an embodiment.

While the invention is susceptible to various modifications and alternative forms, specific embodiments thereof are shown by way of example in the drawings and will herein be described in detail. It should be understood, however, that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

Definitions

The terms used throughout this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

As used herein, the term “xanthophyll carotenoid” generally refers to a naturally occurring or synthetic 40-carbon polyene chain with a carotenoid structure that contains at least one oxygen-containing functional group. The chain may include terminal cyclic end groups. Exemplary, though non-limiting, xanthophyll carotenoids include astaxanthin, zeaxanthin, lutein, echinenone, canthaxanthin, and the like. Non-limiting examples of carotenoids that are not xanthophyll carotenoids include β-carotene and lycopene.

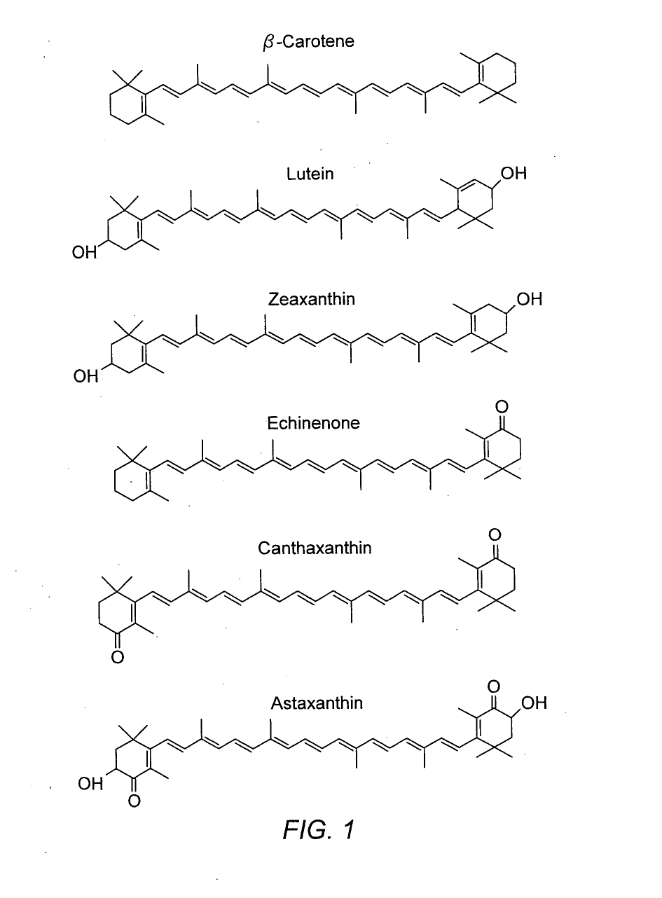

As used herein, terms such as “carotenoid analog” and “carotenoid derivative” generally refer to chemical compounds or compositions derived from a naturally occurring or synthetic carotenoid. Terms such as carotenoid analog and carotenoid derivative may also generally refer to chemical compounds or compositions that are synthetically derived from non-carotenoid based parent compounds; however, which ultimately substantially resemble a carotenoid derived analog. Non-limiting examples of carotenoid analogs and derivatives that may be used according to some of the embodiments described herein are depicted schematically in FIG. 1.

As used herein, the term “organ”, when used in reference to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

As used herein, terms such as “reducing,” “inhibiting,” “ameliorating,” and the like, when used in the context of modulating a physiological or pathological state, generally refer to a net reduction in the magnitude of that state. When used in the context of the effect associated with the administration of the human subject pharmaceutical preparations drug to a human subject, the term(s) generally refer to a net reduction in the severity or seriousness of the oxidative burden experienced by a human subject.

As used herein, the term “Reactive Oxygen Species” (ROS) generally refers to radicals and other non-radical reactive oxygen intermediates that can participate in reactions giving rise to free radicals or that are damaging to organic substrates. Primary reactive oxygen species (ROS) such as superoxide radical, hydrogen peroxide, hydroxyl radicals, and ortho-quinone derivatives of catecholaranes exert their cellular effects by modifying DNA, lipids, and proteins to form secondary electrophiles. Examples of such latter secondary electrophiles include hydroxyalkenals, nucleotide propenals, and hydroxyperoxy fatty acyl chains. The secondary electrophiles are implicated in cellular dysfunction either because they are no longer able to participate in normal cellular activity or because they serve as electron acceptors in oxidative chain reactions that result in the modification of other essential cellular components. Damage caused by the primary and secondary ROS contributes to the etiology of human disease states caused by neuronal ischemia during stroke, post-cardiopulmonary bypass syndrome, brain trauma, and status epilepticus.

As used herein, the term “Reactive Nitrogen Species” (RNS) generally refers to radical nitrogen-based molecules that can act to facilitate nitrosylation reactions.

As used herein, the term “oxidative stress,” generally refers to a pathophysiological state characterized by the generation of ROS in a biological system that exceeds the ability of the system to at least partially neutralize or eliminate them. The imbalance can result from a lack of antioxidant capacity caused by disturbance in production, distribution, or by an overabundance of ROS from an environmental or behavioral stressor. If not regulated properly, the excess ROS can damage the lipids, protein or DNA of a cell, altering its normal function and leading ultimately to the development of certain disease states. The etiology of diseases involving oxidative stress is in part related to the damage caused by the primary and secondary ROS. ROS contribute to the pathogenesis of important human diseases caused by neuronal ischemia during stroke, post-cardiopulmonary bypass syndrome, brain trauma, and status epilepticus. ROS are also involved in cardiac damage induced during ischemic heart disease, renal damage induced by ischemia and toxins as well as in more chronic diseases such as the destruction of neurons in Parkinson's disease, Amyloidosis, Prion disorders, Alzheimer's disease, and other chronic neurodegenerative disorders. Autoimmune diseases such as the destruction of the islets of Langerhans of the endocrine pancreas in Diabetes Mellitus are also encompassed.

As used herein, “redox state” and “redox status” are relative terms that generally refer to the presence and relative concentration of free radicals in a body or body organ. Redox state influences oxidative stress experienced by a body or organ. Systemic or localized changes in oxidative stress, or changes in the levels certain antioxidants, can influence the redox status of the cells or organs.

As used herein, the term “systemically,” when used in the context of a physiological parameter, generally refers to a parameter that affects the entire body of a human subject, or to a particular body system, such as a multi-organ system. For example, the term “systemically reducing oxidative stress in a human subject” generally refers to a net reduction in the oxidative stress throughout the body of a human subject.

As used herein the terms “administration,” “administering,” or the like, when used in the context of providing a pharmaceutical or nutraceutical composition to a human subject generally refers to providing to the human subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, the term “treat” generally refers to an action taken by a caregiver that involves substantially inhibiting, slowing or reversing the progression of a disease, disorder or condition, substantially ameliorating clinical symptoms of a disease disorder or condition, or substantially preventing the appearance of clinical symptoms of a disease, disorder or condition.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, pharmaceutical compositions, formulations and preparations may include pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

The term “pharmaceutically acceptable salt(s)” generally refers to salt(s) prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trisopropylamine, tromethamine, and the like.

Terms such as “in need of treatment,” “in need thereof,” “benefit from such treatment,” or the like, when used in the context of a human subject being administered a pharmacologically active composition, generally refers to a judgment made by an appropriate healthcare provider that an individual requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

By “therapeutically effective amount” is meant an amount of a drug or pharmaceutical composition that will elicit at least one desired biological or physiological response of a cell, a tissue, a system, animal or human that is being sought by a researcher, veterinarian, physician or other caregiver.

By “prophylactically effective amount” is meant an amount of a pharmaceutical composition that will substantially prevent, delay or reduce the risk of occurrence of the biological or physiological event in a cell, a tissue, a system, animal or human that is being sought by a researcher, veterinarian, physician or other caregiver.

The term “pharmacologically inert,” as used herein, generally refers to a compound, additive, binder, vehicle, and the like, that is substantially free of any pharmacologic or “drug-like” activity.

A “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

As used herein the term “antioxidant” may be generally defined as any of various substances (such as beta-carotene, vitamin C, and x-tocopherol) that inhibit oxidation or reactions promoted by Reactive Oxygen Species (ROS) and other radical and non-radical species.

As used herein the term “co-antioxidant” may be generally defined as an antioxidant that is used and that acts in combination with another antioxidant (e.g., two antioxidants that are chemically and/or functionally coupled, or two antioxidants that are combined and function with each another in a pharmaceutical preparation). The effects of co-antioxidants may be additive (i.e., the anti-oxidative potential of one or more anti-oxidants acting additively is approximately the sum of the oxidative potential of each component anti-oxidant) or synergistic (i.e., the anti-oxidative potential of one or more anti-oxidants acting synergistically may be greater than the sum of the oxidative potential of each component anti-oxidant).

Compounds described herein embrace isomers mixtures, racemic, optically active, and optically inactive stereoisomers and compounds.

General Description

The presently described embodiments provide for novel methods for the treatment or prophylaxis in patients of pathological conditions involving elevated redox state and oxidative stress, comprising administering to said patients a therapeutically or prophylactically effective amount of a xanthophyll carotenoid, or a carotenoid derivative or analog.

The presently described embodiments also provide pharmaceutical compositions comprising a therapeutically or prophylactically effective amount of xanthophyll carotenoids, or a carotenoid derivative or analogs, and a pharmaceutically acceptable carrier in unit dosage form.

Clinical studies have demonstrated a correlation between elevated oxidative stress and the appearance of certain pathological states. Oxidative stress is imposed on cells or tissues as a result of one of three factors: 1) an increase in oxidant generation, 2) a decrease in antioxidant protection, or 3) a failure to repair oxidative damage. Cell damage is induced by reactive oxygen species (ROS). ROS are either free radicals, reactive anions containing oxygen atoms, or molecules containing oxygen atoms that can either produce free radicals or are chemically activated by them. Typically, the primary source of ROS in cells is aerobic respiration, although ROS are also produced by peroxisomal β-oxidation of fatty acids, microsomal cytochrome P450 metabolism of xenobiotic compounds, stimulation of phagocytosis by pathogens or lipopolysaccharides, arginine metabolism, and tissue specific enzymes. Under normal conditions, ROS are cleared from the cell by the action of superoxide dismutase (SOD), catalase, or glutathione (GSH) peroxidase. The main damage to cells results from the ROS-induced alteration of macromolecules such as polyunsaturated fatty acids in membrane lipids, essential proteins, and DNA.

It has been noted that certain organ systems are predisposed to greater levels of oxidative or nitrosative stress. Those organ systems most susceptible to damage are the pulmonary system (exposed to high levels of oxygen), the central nervous system (CNS) (exhibits intense metabolic activity yet has lower levels of endogenous antioxidants), the eye (constantly exposed to damaging UV light), circulatory system (subject to fluctuating oxygen and nitric oxide levels) and reproductive systems (at risk from the intense metabolic activity of sperm cells). However, situations that impose oxidative stress on cells and various tissues have been observed in most organ systems. Ordinary practitioners of the art would readily recognize such situations.

Proteins, lipids, and nucleic acids (including DNA) are among the biomolecules that can be damaged by oxidative stress. Oxidative damage to proteins can be brought about by the oxidation of cysteine with formation of disulphide, the oxidation of methionine with formation of sulphoxide and sulphone, and the oxidation of tryptophan and formation of N-formyl kynurenine and kynurenine. Oxidative damage to proteins also can occur by the hydroperoxidation of valine, leucine or lysine. Metal-catalyzed oxidation of histidine and formation of 2- and 8-oxohistidine also can cause oxidative damage to proteins. Still further, oxidative damage also can occur by tyrosine dimerization with resultant protein aggregation. Oxidative damage to proteins can occur by the formation of carbonyls, the formation of adipic semi-aldehyde from lysine, the formation of L-DOPA from tyrosine, the formation of alkyl- (chloro- or bromo-) tyrosine, the formation of nitrotyrosine, the formation of para-, meta- and ortho-tyrosine from phenylalanine, and the formation of neoepitopes on oxidized proteins. Oxidative changes of proteins can give rise to new formations that are recognized as foreign by the immune system and elicit an immune response.

Oxidative damage to lipids may occur by formation of aldehydes (e.g., malondialdehyde and 4-HNE), pentane and ethane, 2,3 trans-conjugated dienes, isoprostane, cholesteroxides, lipofuiscin, and isolevuglandin adducts, which can cause protein-DNA and protein-protein cross-linking.

Oxidative damage to nucleic acids can occur by the formation of 8-nitroguanine, 8-chloroadenine, adenine N¹-oxide, and tandem base products selected from the group consisting of thymine-guanine ([5-methyl]-8), guanine-thymine(8-[5-methyl]), 6-hydroxythymine-guanine(5-8), guanine-6-hydroxythymine(8-5), adenine-thymine(8-[5-methyl]), thymine-adenine([5-methyl]-8), cytosine-guanine(5-8), and dihydrouracil-guanine(5-8). Oxidative damage to nucleic acids also can occur by the oxidation of: thymines (e.g., at the 5,6-double bond or at the 5-methyl group); cytosines at the 5,6-double bond; 5-methylcytosines; guanines to 8-oxoguanine; guanines to 2,6-diamino-4-hydroxy-formamidopyrimidine (fapyG); adenines to 4,6-diamino-formamidopyrimidine (fapyA); adenines to 8-hydroxyadenine; adenines to 2-hydroxyadenine; and, adenines to adenine N.sup.6-hydroxylamine. The resultant DNA structural and functional alterations include, but are not limited to, point mutations, replicative blocks, deletions, microsatellite instability/loss of heterozygosity, and epigenetic effects.

Biomolecular damage caused by oxidative stress often leads to the induction and propagation of oxidative stress-associated conditions, including, but not limited to, diseases of the blood, brain/nervous system, breast (e.g., invasive ductal carcinoma and cancer), cardiovascular system (e.g., coronary heart disease), colon (e.g., colorectal cancer), kidney (e.g., renal cell carcinoma and reperfusion injury), liver, respiratory system, skin, and stomach (e.g., H. pylori infection and cancer). Additionally, oxidative stress-associated diseases include diabetes mellitus (both insulin-dependent diabetes mellitus (IDDM) and non-IDDM), Down's Syndrome, exposure toxicity, gynecological diseases, gastrointestinal system (e.g., inflammatory bowel disease), metabolic syndrome, pancreatitis, preeclampsia, prostate cancer, rheumatoid arthritis, systemic lupus erythematosus (SLE), and viral diseases (e.g., HIV). Blood diseases include acute lymphoblastic leukemia and Fanconi's anemia. Brain/nervous system disease include Alzheimer's disease, amyotrophic lateral sclerosis, cerebral amyloid angiopathy, Charcot Marie Tooth, dementia with Lewy bodies, Friedreich ataxia multiple sclerosis, and Parkinson's disease. Cardiovascular diseases include atherosclerosis, hypertension, thrombosis, and heart disease, such as coronary heart disease. Liver diseases caused by oxidative stress include, but are not limited to, chronic hepatitis, hepatitis C, hepatoblastoma, alcoholic liver disease, primary billiary cirrhosis, and hepatacellular carcinoma. Respiratory system diseases caused by oxidative stress include, but are not limited to, acute respiratory distress syndrome, asthma, chronic obstructive pulmonary dysfunction (COPD), cystic fibrosis, obstructive sleep apnea, squamous cell carcinoma, and, small cell carcinoma. Skin diseases caused by oxidative stress include, but are not limited to atopic dermatitis, skin neoplasma, skin wrinkling, pre-cancerous skin changes, viteligo, and psoriasis. Other oxidative stress-associated conditions include, but are not limited to, cancer generally and aging. More detailed descriptions of the foregoing conditions can be found in, for example, Thomas E. Andreoli, M. D. (Editor), “Cecil Essentials of Medicine,” 3.sup.rd Ed. (Harcourt Brace & Company, Philadelphia, Pa., 1993).

The methods described herein include administering to a human subject who would benefit from reduced systemic and/or target organ oxidative stress an effective amount of a pharmaceutically acceptable formulation that includes a xanthophyll carotenoid or a synthetic analog or derivative of a xanthophyll carotenoid. In an embodiment, the formulation may include astaxanthin, lutein and/or zeaxanthin or a structural analog or a derivative thereof. In an embodiment, the formulation may include homochiral (“chiral”) astaxanthin, or a synthetic analog or derivative of a homochiral astaxanthin. In an embodiment, the formulation may include mixtures of varying proportions of different homochiral forms of astaxanthin. In an embodiment, a synthetic analog or derivative of a chiral astaxanthin may be administered to a human subject, wherein the synthetic chiral asatxanthin, when present in the human subject's body, undergoes chemical or enzymatic breakdown, wherein at least one breakdown product is chiral asatxanthin. In certain preferred embodiments, the various synthetic and/or naturally occurring forms of astaxanthin (stereoisomers, geometric isomers, monoesters, diesters) may be administered to a human subject to achieve the intended purpose. The term “astaxanthin” therefore includes its various chemical forms, and may include a certain preferred isomeric or ester form for a particular use. Exemplary though non-limiting xanthophyll carotenoids or structural derivatives or analogs thereof that may be suitable for use in the embodiments disclosed herein are depicted schematically in FIG. 1

The pharmaceutical preparation may be administered orally, in the form of a tablet, a capsule, an emulsion, a liquid, or the like. Alternatively, the pharmaceutical preparation may be administered via a parenteral route. A more detailed description of the types of pharmaceutical preparations that may be suitable for some embodiments is described in below. Some embodiments may be particularly suited timed or sustained release pharmaceutical preparations, in which the preparation is adapted to deliver a known dosage of xanthophyll carotenoids or synthetic derivatives or analogs thereof at or over a predetermined time.

In addition to carotenoids or structural analogs or derivatives thereof, a pharmaceutical composition may further include one or more co-antioxidant compounds. Exemplary, though non-limitive, co-antioxidant compounds that may be suitable for inclusion in a pharmaceutical preparation together with the xanthophyll carotenoids disclosed herein, or structural analogs or derivatives thereof, may include ascorbic acid or vitamin-E (α-tocopherol). In an embodiment, co-antioxidant compounds may be covalently linked to the xanthophyll carotenoids or structural analogs or derivatives. Alternatively, co-antioxidant compounds may be mixed with the xanthophyll carotenoids or structural analogs or derivatives.

Administration of xanthophyll carotenoids or a synthetic analogs or derivatives thereof according to the preceding embodiments may be used to treat any one of a plurality of diseases, disorders or conditions associated with oxidative stress.

In some embodiments, the administration of xanthophyll carotenoids or structural analogs or derivatives of carotenoids by one skilled in the art—including consideration of the pharmacokinetics and pharmacodynamics of therapeutic drug delivery—is expected to inhibit and/or ameliorate disease conditions associated with administering xanthophyll carotenoid or a synthetic analog or derivative thereof to a human subject, including but not limited to the production of oxidized lipids, isoprostanes and LDL. In some of the foregoing embodiments, analogs or derivatives of carotenoids administered to a human subject may be adapted to be at least partially water-soluble.

The methods provided for herein can be used to treat non-central nervous system disorders such as rheumatoid arthritis, cataract, Down syndrome, cystic fibrosis, diabetes, acute respiratory distress syndrome, asthma, post-surgical neurological dysfunction, amyotrophic lateral sclerosis, atherosclerotic cardiovascular disease, hypertension, post-operative restenosis, pathogenic vascular smooth muscle cell proliferation, pathogenic intra-vascular macrophage adhesion, pathogenic platelet activation, pathogenic lipid peroxidation, myocarditis, stroke, multiple organ dysfunction, complication resulting from inflammatory processes, AIDS, cancer, aging, bacterial infection, sepsis; viral disease, such as AIDS, hepatitis C, an influenza and a neurological viral disease, all of which were previously shown to be linked to oxidative stress.

The methods provided for herein can also be used to treat a central nervous system disorder characterized by oxidative stress, such as, neurodegenerative disorders, Parkinson's disease, Alzheimer's disease, Creutzfeldt-Jakob disease, cerebral ischemia, multiple sclerosis, degenerative diseases of the basal ganglia, motoneuron diseases, scrapies, spongiform encephalopathy, neurological viral diseases, chronic fatigue syndrome (CFS), motoneuron diseases, post-surgical neurological dysfunction and loss or memory impairment, all of which were previously shown to be linked to oxidative stress.

Carotenoids and the Preparation and Use Thereof

In some embodiments, a composition may include one or more carotenoids, or synthetic derivatives or analogs thereof having the general structure:

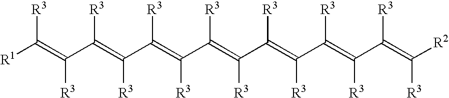

where each R³ is independently hydrogen or methyl, and where each R¹ and R² are independently: 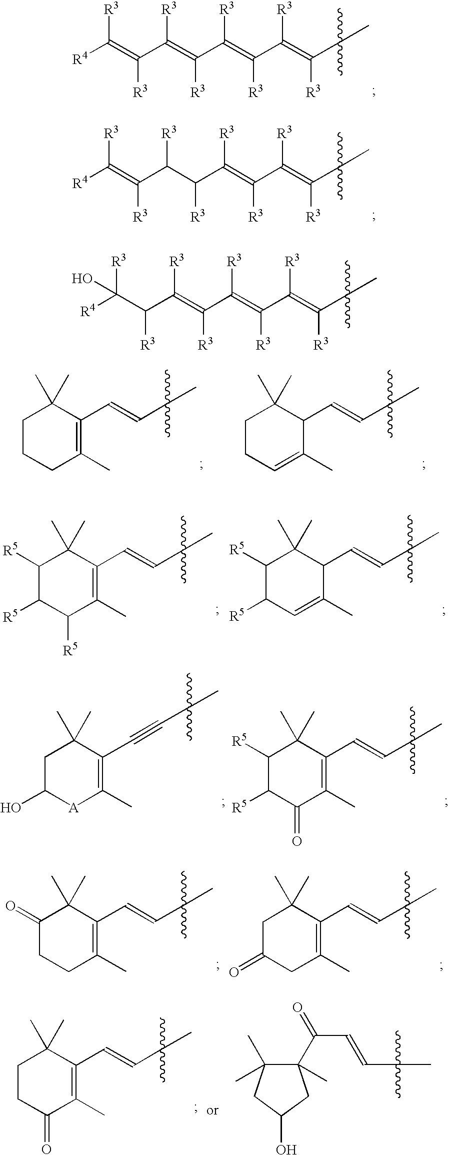
where R⁴ is hydrogen, methyl, or —CH₂OH; and where each R⁵ is independently hydrogen or —OH. Sources of some of these carotenoids can be found, for example, in the reference “Key to Cartenoids”, Otto Straub, 2^{nd} Ed., Birkhauser Verlag, Boston, 1987, which is incorporated herein by reference.

In some embodiments, a composition used for the methods described herein may include carotenoids, or synthetic derivatives or analogs thereof having the structure:

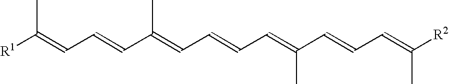

where each R¹ and R² is independently:

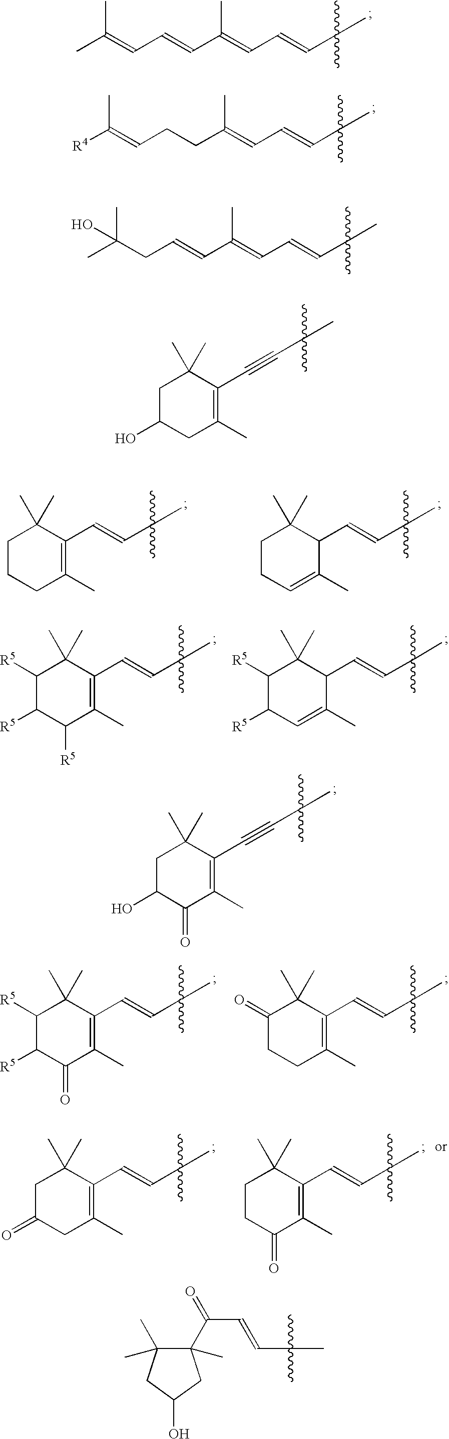

where R⁴ is hydrogen, methyl, or —CH₂OH; and where each R⁵ is independently hydrogen or —OH.

In some embodiments, a composition used for the methods described herein may include carotenoids having the general structure:

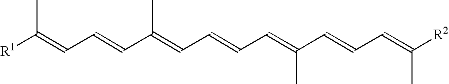

where each R¹ and R² is independently:

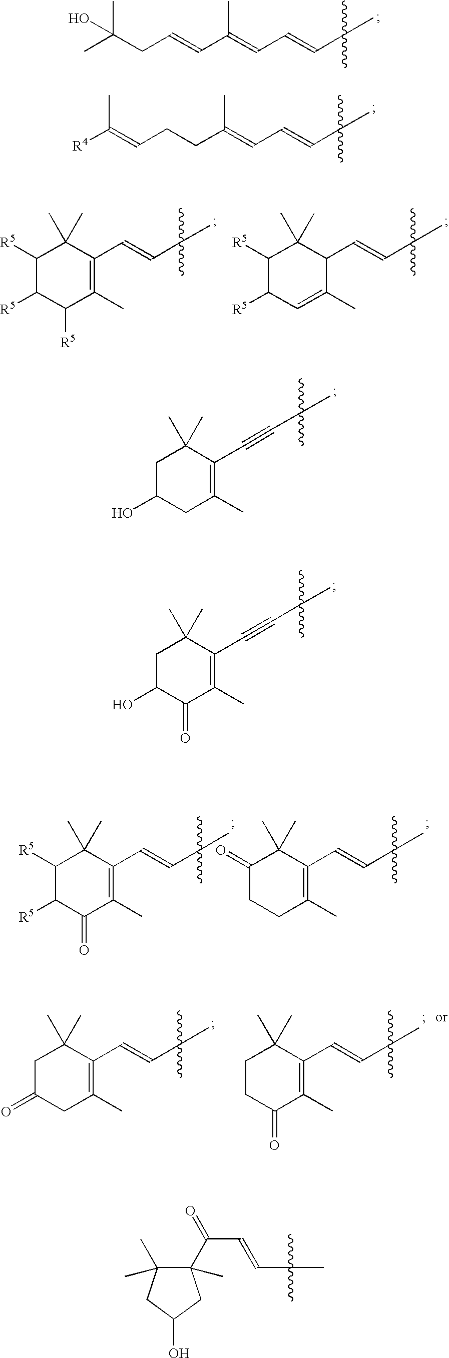

where R⁴ is —CH₂—OH; and where each R⁵ is independently hydrogen or —OH.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, flavonoids, flavonoid analogs, or flavonoid derivatives), or combinations thereof derivatives or analogs of carotenoids. Flavonoids may include, for example, quercetin, xanthohumol, isoxanthohumol, or genistein. Vitamin E may generally be divided into two categories including tocopherols having a general structure 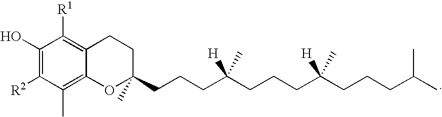
Alpha-tocopherol is used to designate when R¹=R²=CH₃. Beta-tocopherol is used to designate when R¹=CH₃ and R²=H. Gamma-tocopherol is used to designate when R¹=H and R²=CH₃. Delta-tocopherol is used to designate when R¹=R²=H.

The second category of Vitamin E may include tocotrienols having a general structure 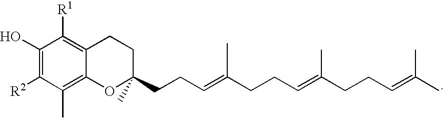
Alpha-tocotrienol is used to designate when R¹=R²=CH₃. Beta-tocotrienol is used to designate when R¹=CH₃ and R²=H. Gamma-tocotrienol is used to designate when R¹=H and R²=CH₃. Delta-tocotrienol is used to designate when R¹=R²=H.

Quercetin, a flavonoid, has the structure 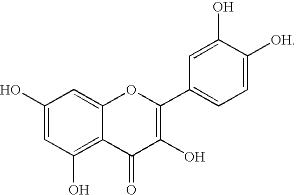
In some embodiments, one or more co-antioxidants may be coupled to a carotenoid or carotenoid derivative or analog. Derivatives of one or more carotenoid analogues may be formed by coupling one or more free hydroxy groups of the co-antioxidant to a portion of the carotenoid.

Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

In some embodiments, carotenoid analogs or derivatives may have increased water solubility and/or water dispersibility relative to some or all known naturally occurring carotenoids. Contradictory to previous research, improved results are obtained with derivatized carotenoids relative to the base carotenoid, wherein the base carotenoid is derivatized with substituents including hydrophilic substituents and/or co-antioxidants.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a human subject or patient.

In some embodiments, a chemical compound including a carotenoid derivative or analog may have the general structure: 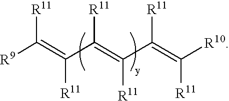
Each R¹¹ may be independently hydrogen or methyl. R⁹ and R¹⁰ may be independently H, an acyclic alkene with one or more substituents, or a cyclic ring including one or more substituents. In some embodiments, y may be 5 to 12. In some embodiments, y may be 3 to 15. In certain embodiments, the maximum value of y may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be included in a pharmaceutical composition.

In some embodiments, methods for systemically inhibiting or reducing oxidative stress in the body or in a body organ of a human subject, or for modulating the redox status of a body organ may include administering to the human subject an effective amount of a pharmaceutically acceptable formulation including one or more synthetic analogs or derivatives of a carotenoid. The synthetic analog or derivative of the carotenoid may have the structure

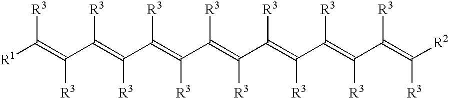

where each R³ is independently hydrogen or methyl, and where each R¹ and R² is independently: 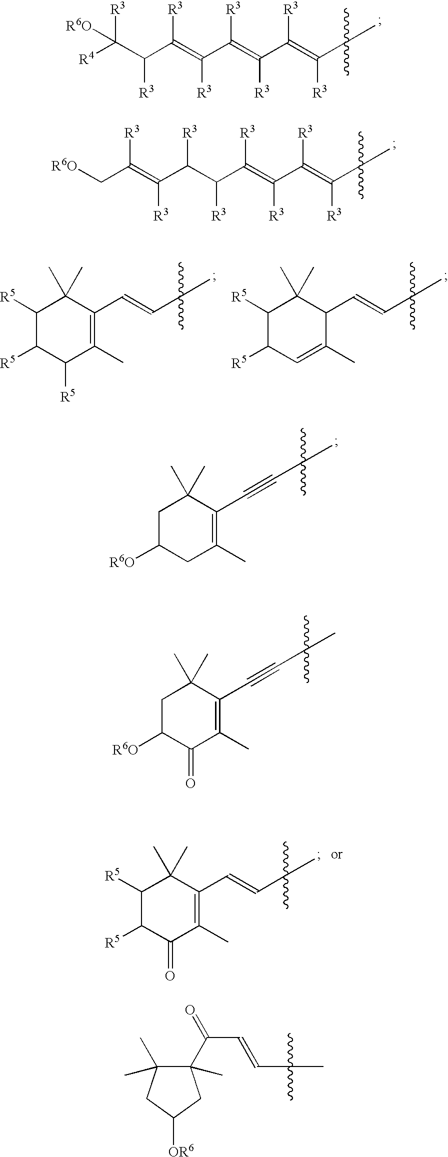
where R⁴ is hydrogen or methyl; where each R⁵ is independently hydrogen, —OH, or —OR⁶ wherein at least one R⁵ group is —OR⁶; wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may be used for systemically inhibiting or reducing oxidative stress in the body or in a body organ of a human subject, or for modulating the redox status of a body organ.

Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids include, but are not limited to, quercetin, xanthohumol, isoxanthohumol, or genistein. Selection of the co-antioxidant should not be seen as limiting for the therapeutic application of the current invention.

In some embodiments, methods for systemically inhibiting or reducing oxidative stress in the body or in a body organ of a human subject, or for modulating the redox status of a body organ may include administering to the human subject an effective amount of a pharmaceutically acceptable formulation including one or more synthetic analogs or derivatives of a carotenoid. The synthetic analog or derivative of the carotenoid may have the structure

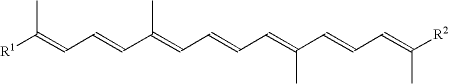

where each R¹ and R² is independently:

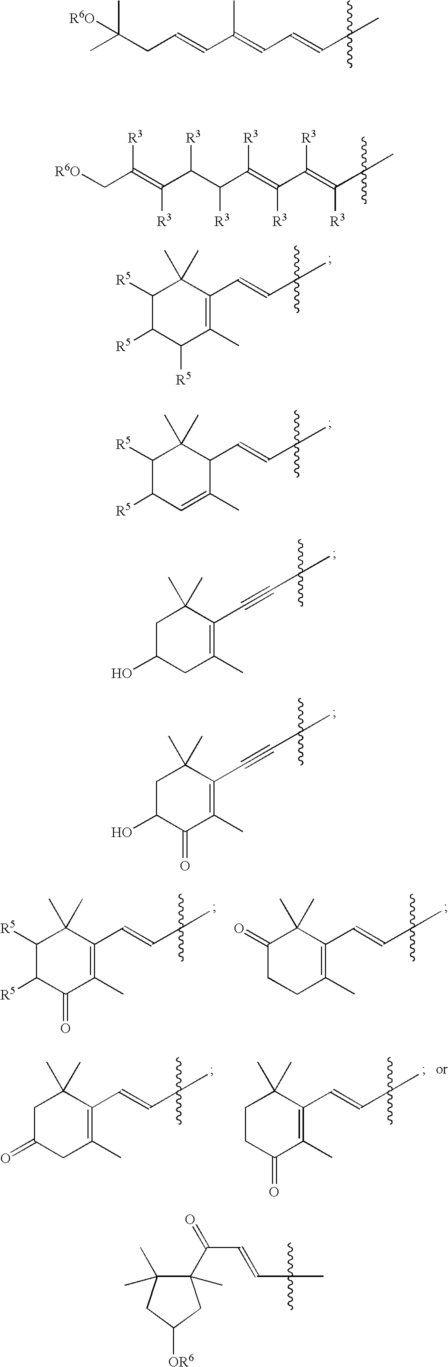

where each R⁵ is independently hydrogen, —OH, or —OR⁶ wherein at least one R⁵ group is —OR⁶; wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂, -aryl-CO₂, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

In some embodiments, methods for systemically inhibiting or reducing oxidative stress in the body or in a body organ of a human subject, or for modulating the redox status of a body organ may include administering to the human subject an effective amount of a pharmaceutically acceptable formulation including one or more synthetic analogs or derivatives of a carotenoid. The synthetic analog or derivative of the carotenoid may have the structure 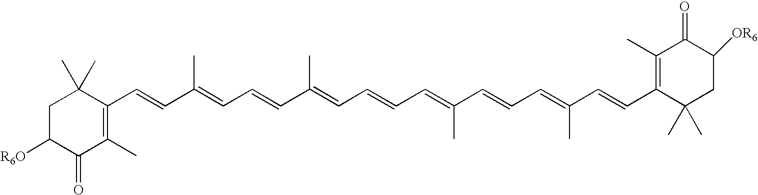
wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂), —CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

In some embodiments, methods for systemically inhibiting or reducing oxidative stress in the body or in a body organ of a human subject, or for modulating the redox status of a body organ may include administering to the human subject an effective amount of a pharmaceutically acceptable formulation including one or more synthetic analogs or derivatives of a carotenoid. The synthetic analog or derivative of the carotenoid may have the structure 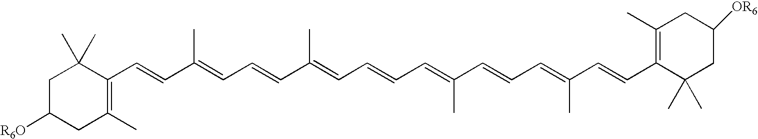
wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

In some embodiments, methods for systemically inhibiting or reducing oxidative stress in the body or in a body organ of a human subject, or for modulating the redox status of a body organ may include administering to the human subject an effective amount of a pharmaceutically acceptable formulation including one or more synthetic analogs or derivatives of a carotenoid. The synthetic analog or derivative of the carotenoid may have the structure 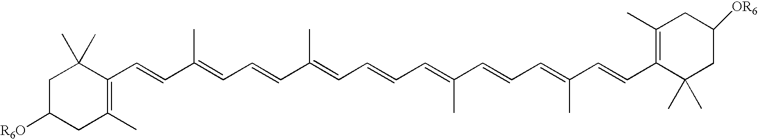
wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

When R⁶ is an amino acid derivative or a peptide, coupling of the amino acid or the peptide is accomplished through an ester linkage. The ester linkage may be formed between a free hydroxyl of the xanthophyll carotene and the carboxylic acid of the amino acid or peptide. When R⁹ is an amino acid derivative or a peptide, coupling of the amino acid or the peptide is accomplished through an amide linkage. The amide linkage may be formed between the terminal carboxylic acid group of the linker attached to the xanthophyll carotene and the amine of the amino acid or peptide.

When R⁶ is a sugar, R⁶ includes, but is not limited to the following side chains:

—CH₂—(CHOH)ₙ—CO₂H;

—CH₂—(CHOH)ₙ—CHO;

—CH₂—(CHOH)ₙ—CH₂OH;

—CH₂—(CHOH)ₙ—C(O)—CH₂OH; 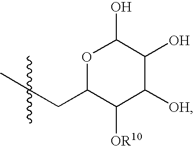
where R¹⁰ is hydrogen or 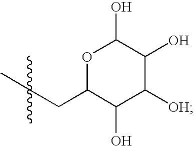 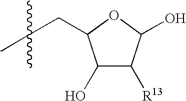
where R¹³ is hydrogen or —OH.

When R⁶ is a nucleoside, R⁶ may have the structure:

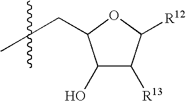

where R¹² is a purine or pyrimidine base, and R¹³ is hydrogen or —OH.

In some embodiments, the carotenoid analog or derivative may have the structures 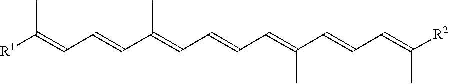
where each R¹ and R² are independently: 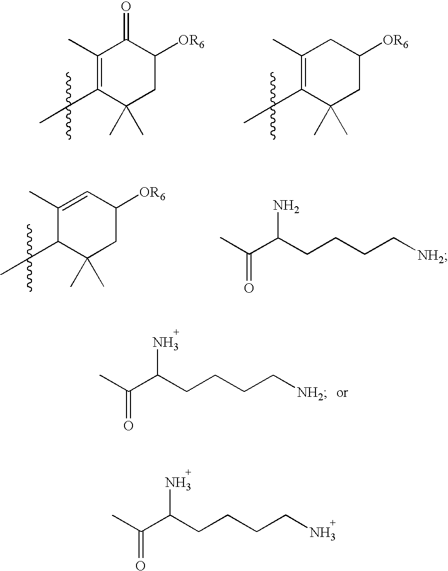
where R⁶ is

In some embodiments, the carotenoid analog or derivative may have the structures 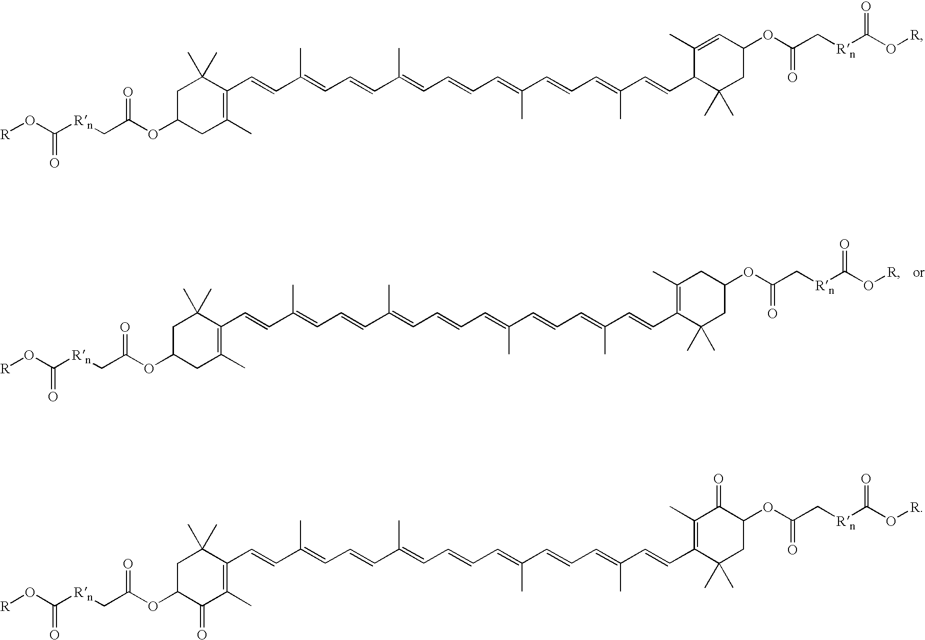
Each R′ₙ may be independently —(CH₂)ₙ—; where n=1-10. Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

In some embodiments, the carotenoid analog or derivative may have the structures 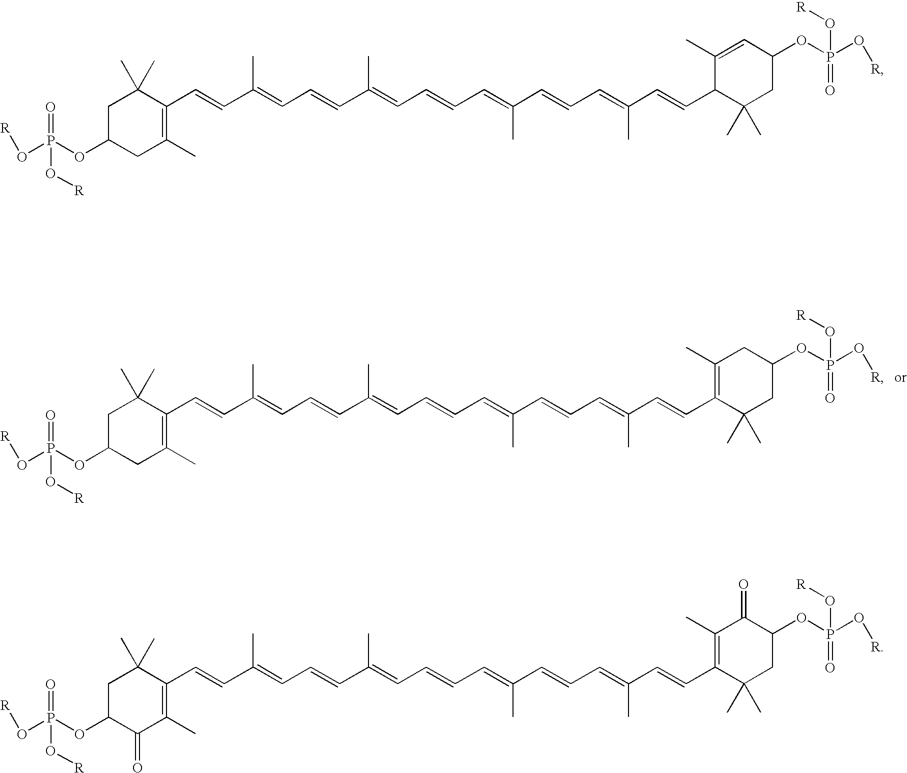
Each R may be independently H, alkyl, aryl, benzyl, Group IA metal (e.g., sodium), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. When R includes Vitamin C, Vitamin C analogs, or Vitamin C derivatives, some embodiments may include carotenoid analogs or derivatives having the structure 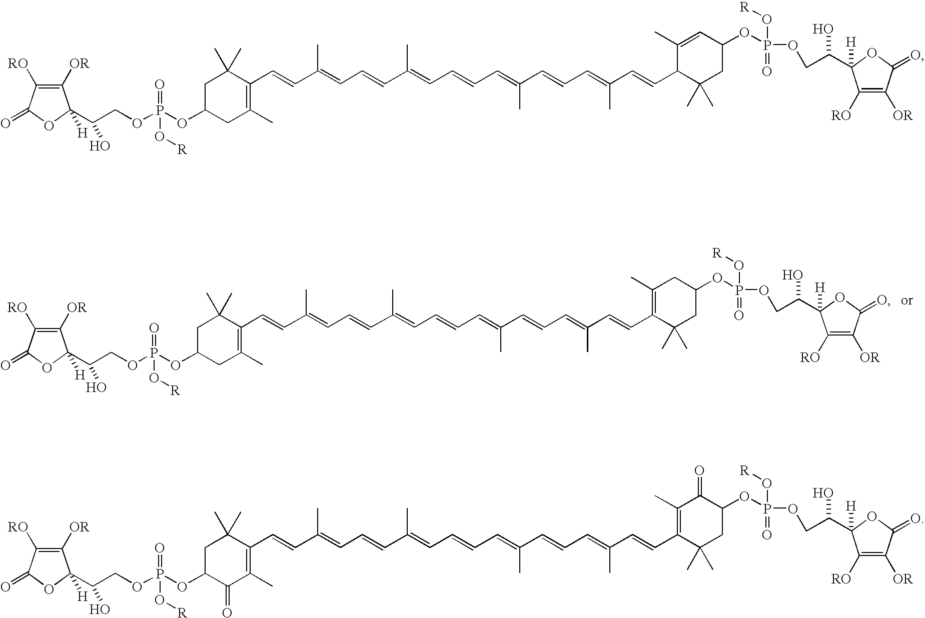
Each R may be independently H, alkyl, aryl, benzyl, or Group IA metal.

In some embodiments, the carotenoid derivative may have the structure:

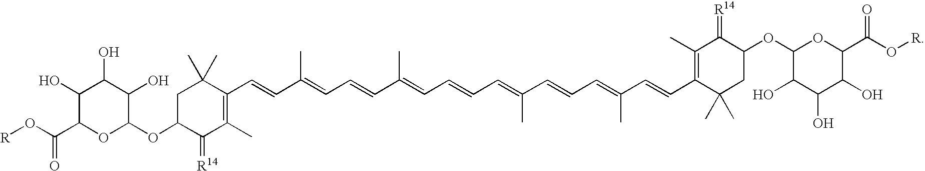

Each R¹⁴ may be independently O or H₂. Each R may be independently H, alkyl, benzyl, Group IA metal, co-antioxidant, or aryl.

Specific examples of carotenoid derivatives include, but are not limited to, the following compounds:

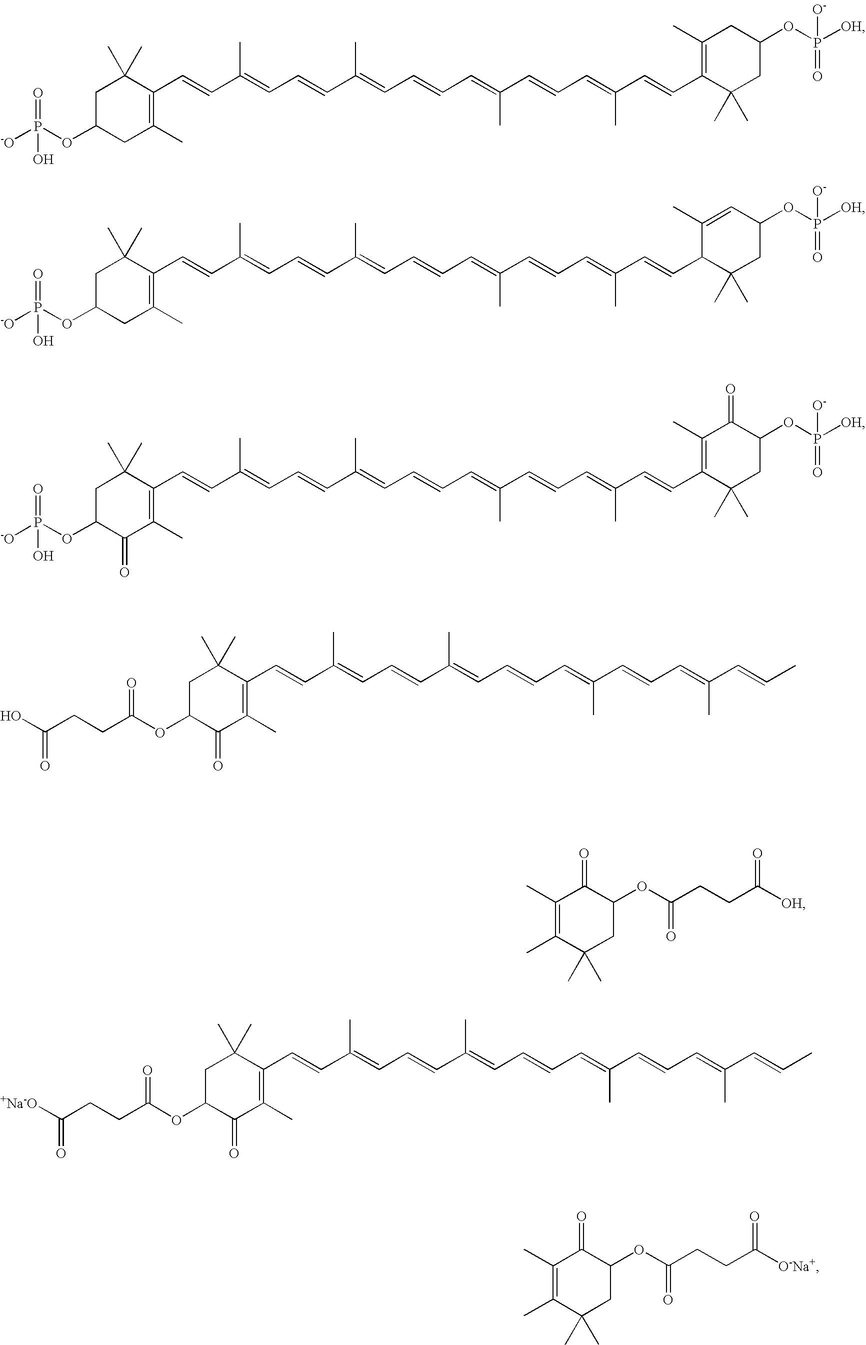

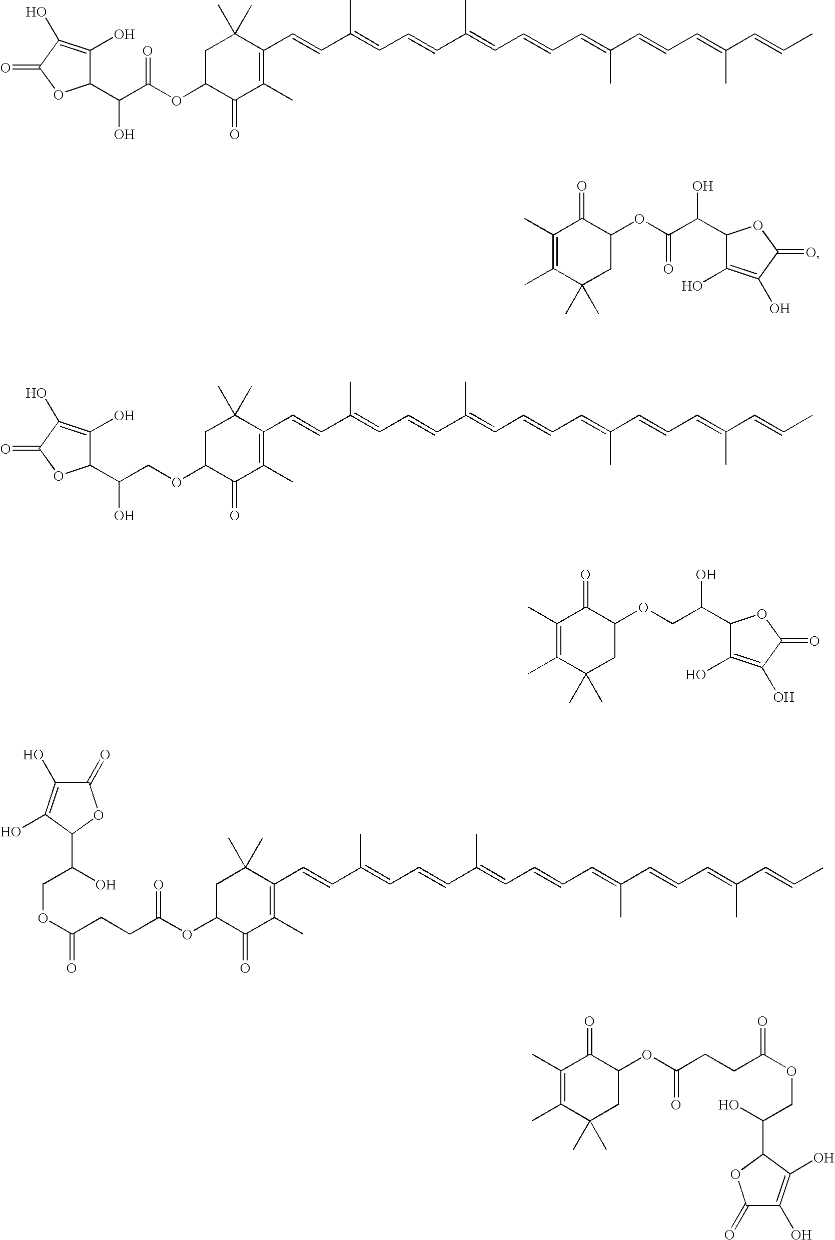

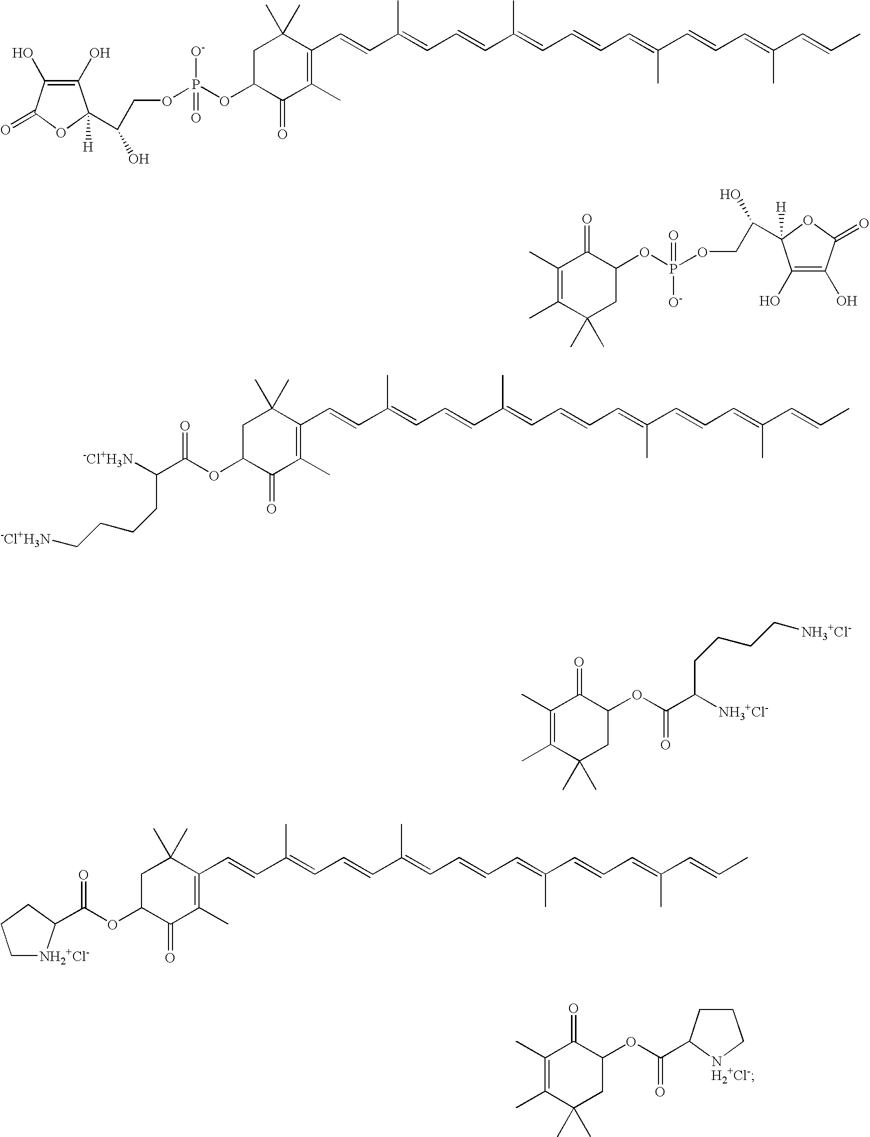

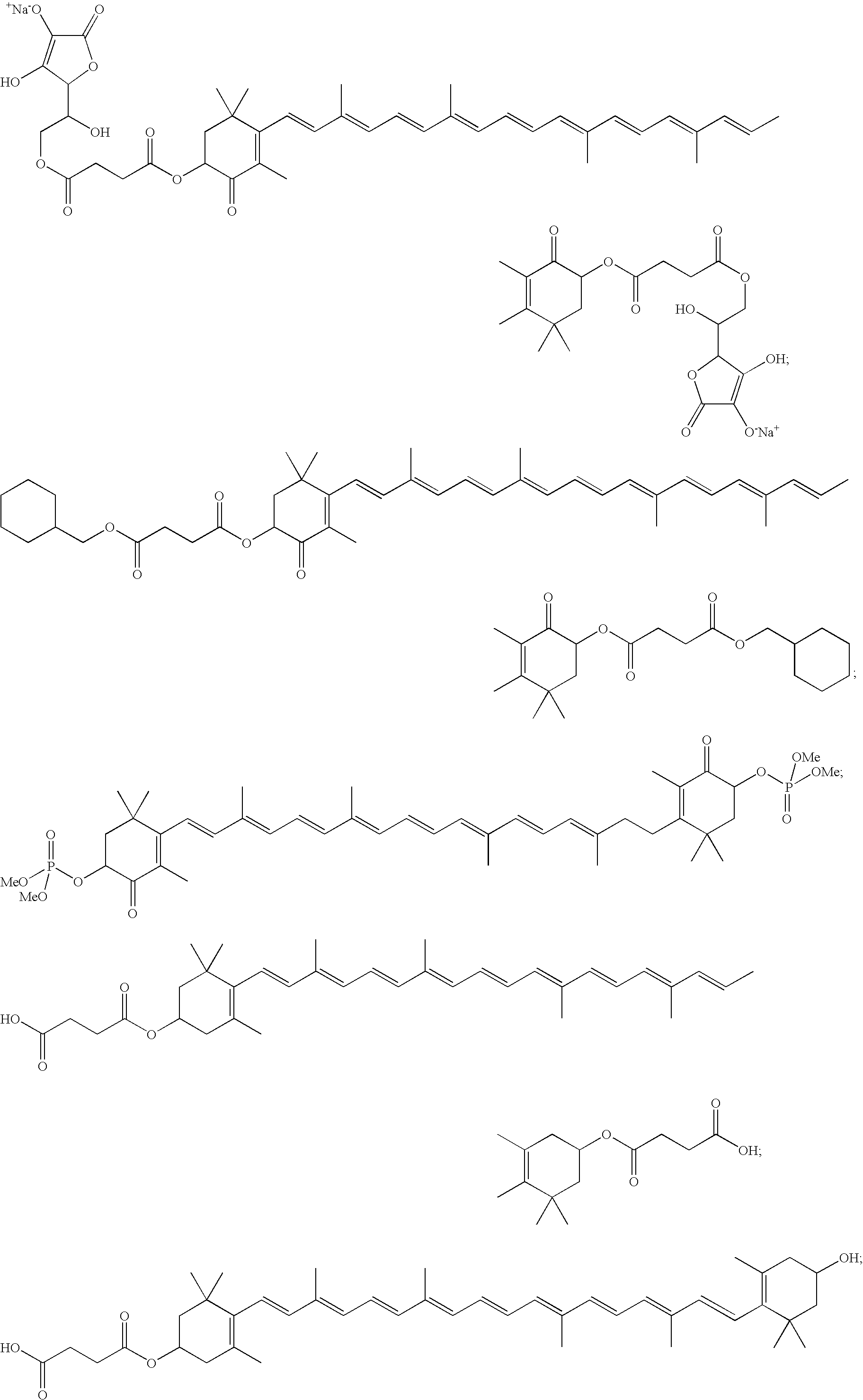

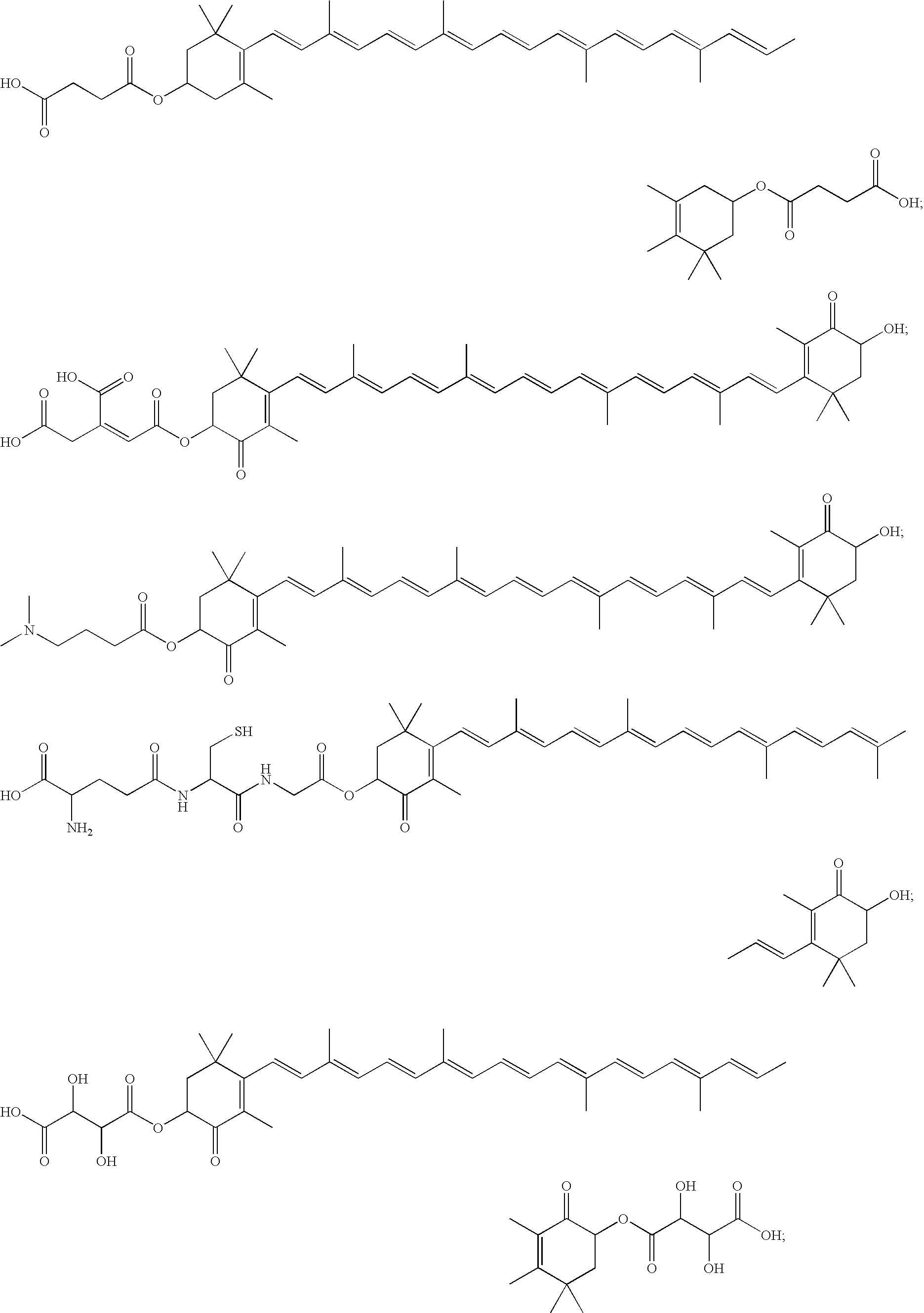

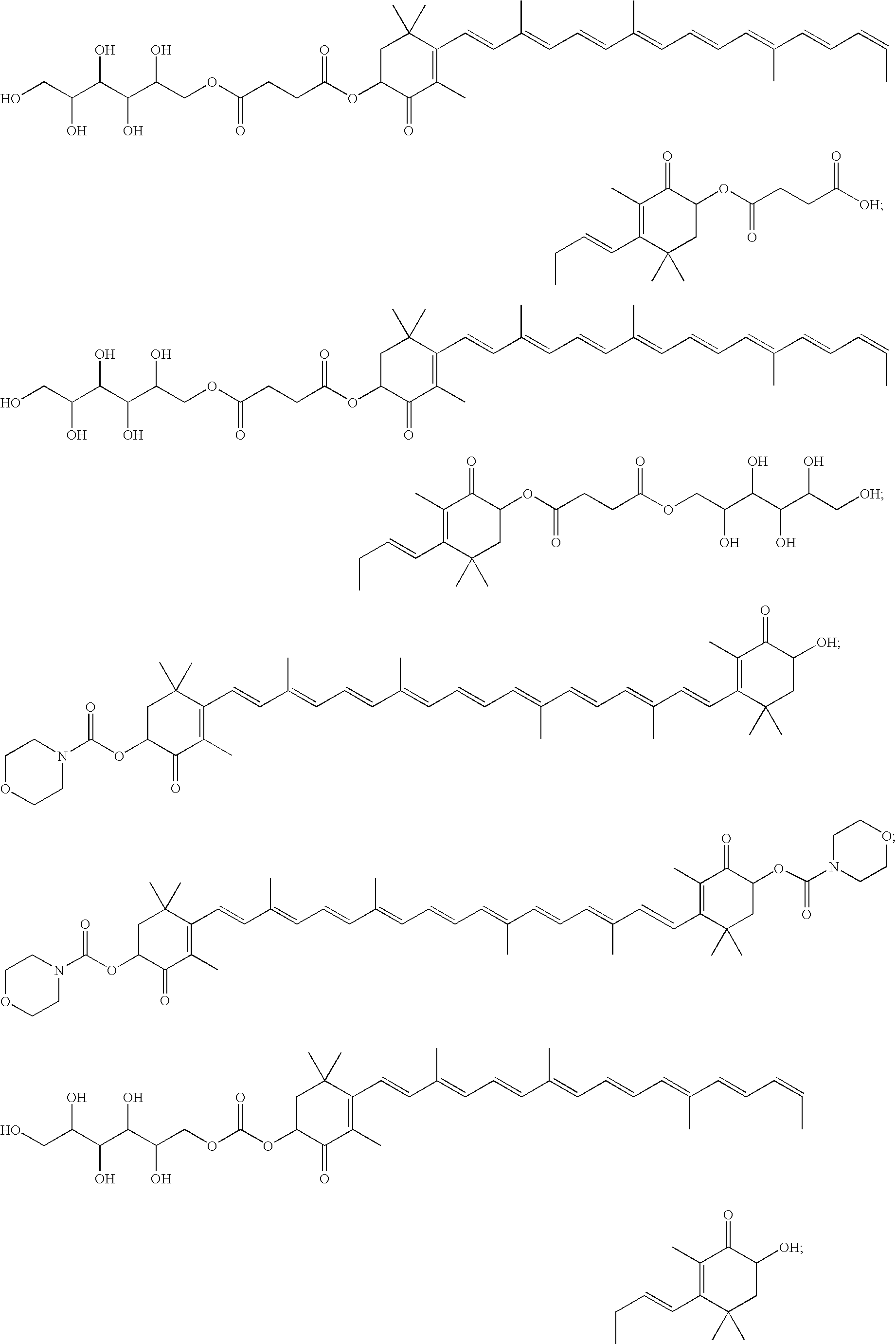

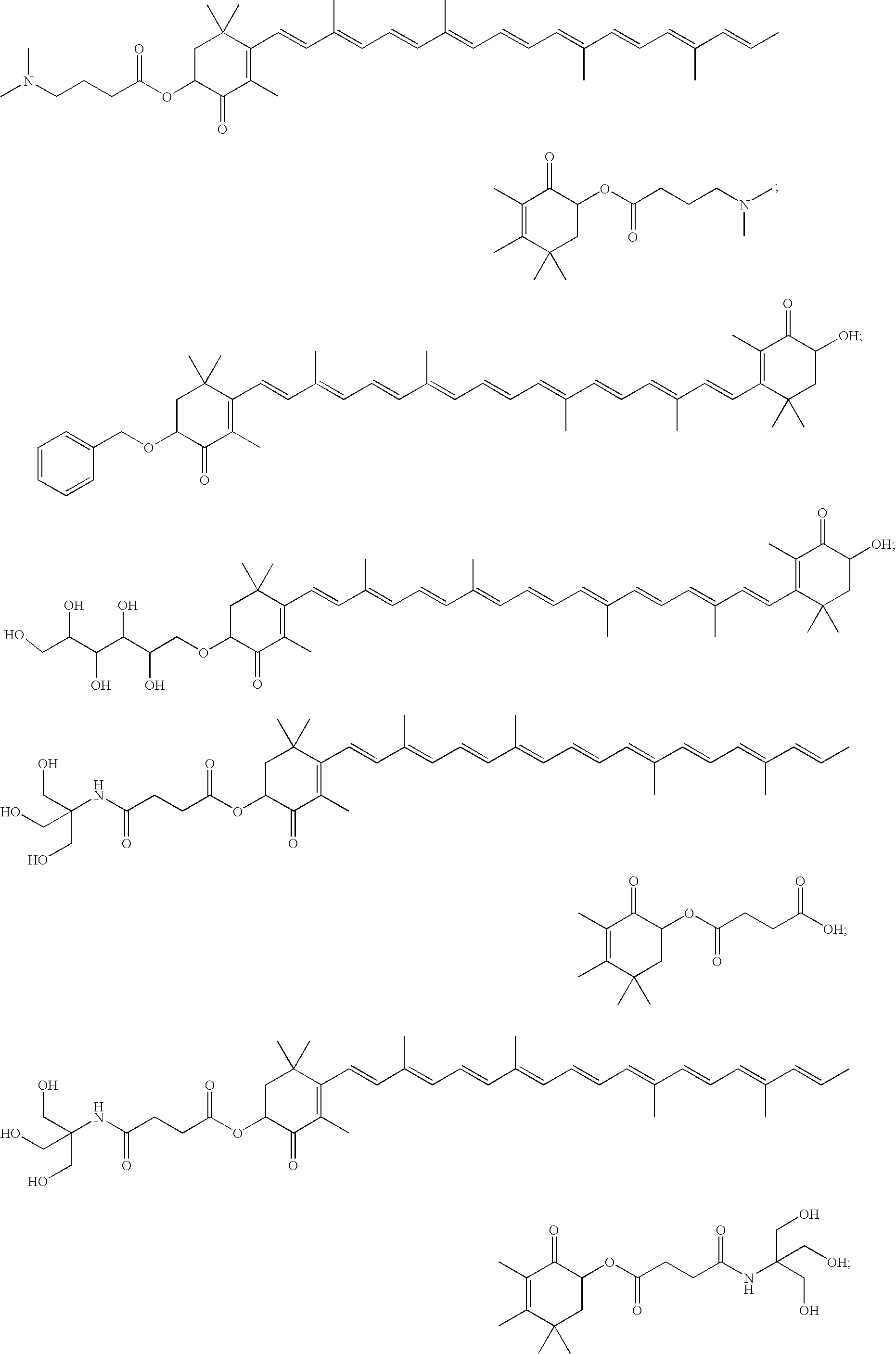

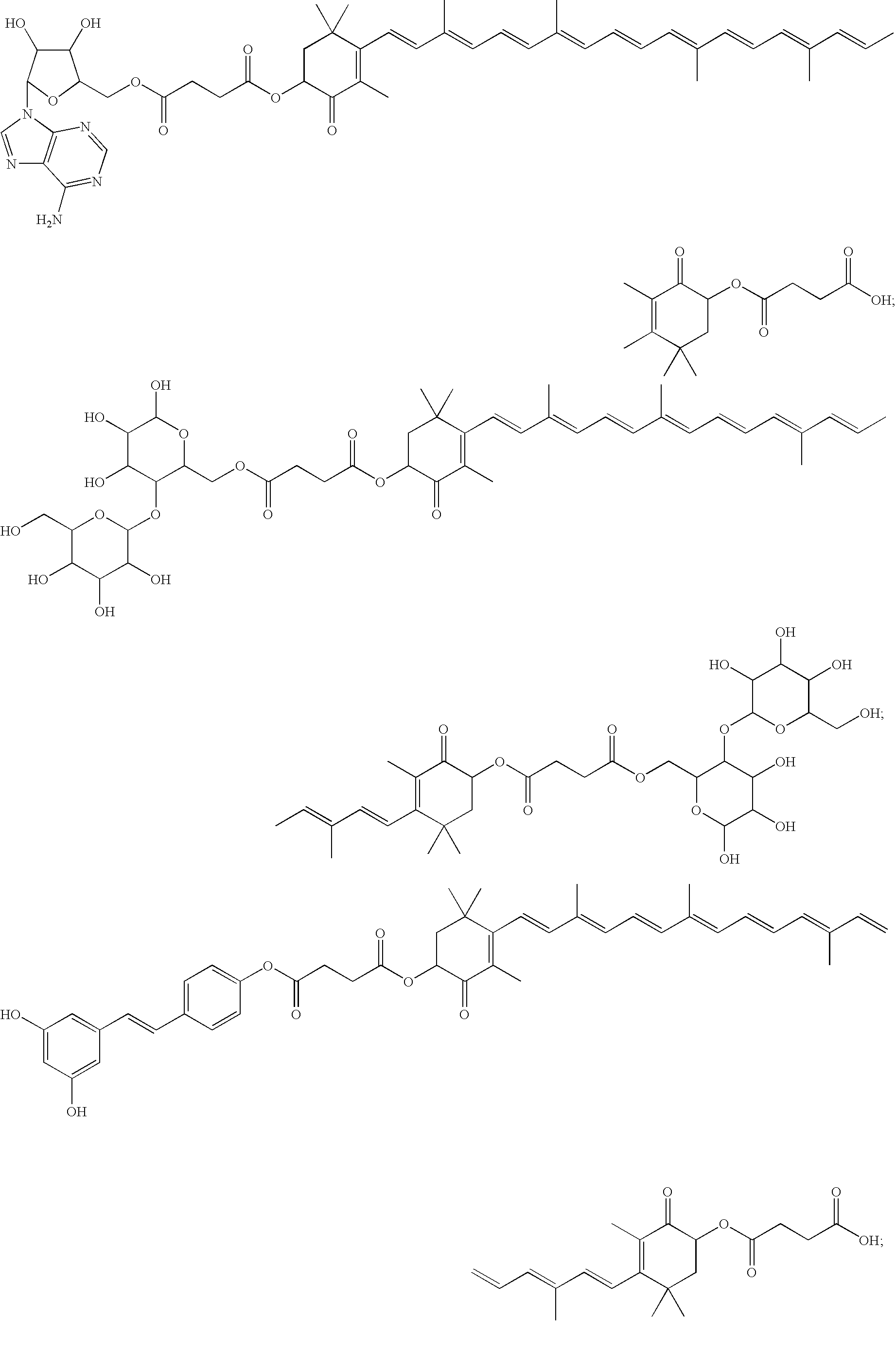

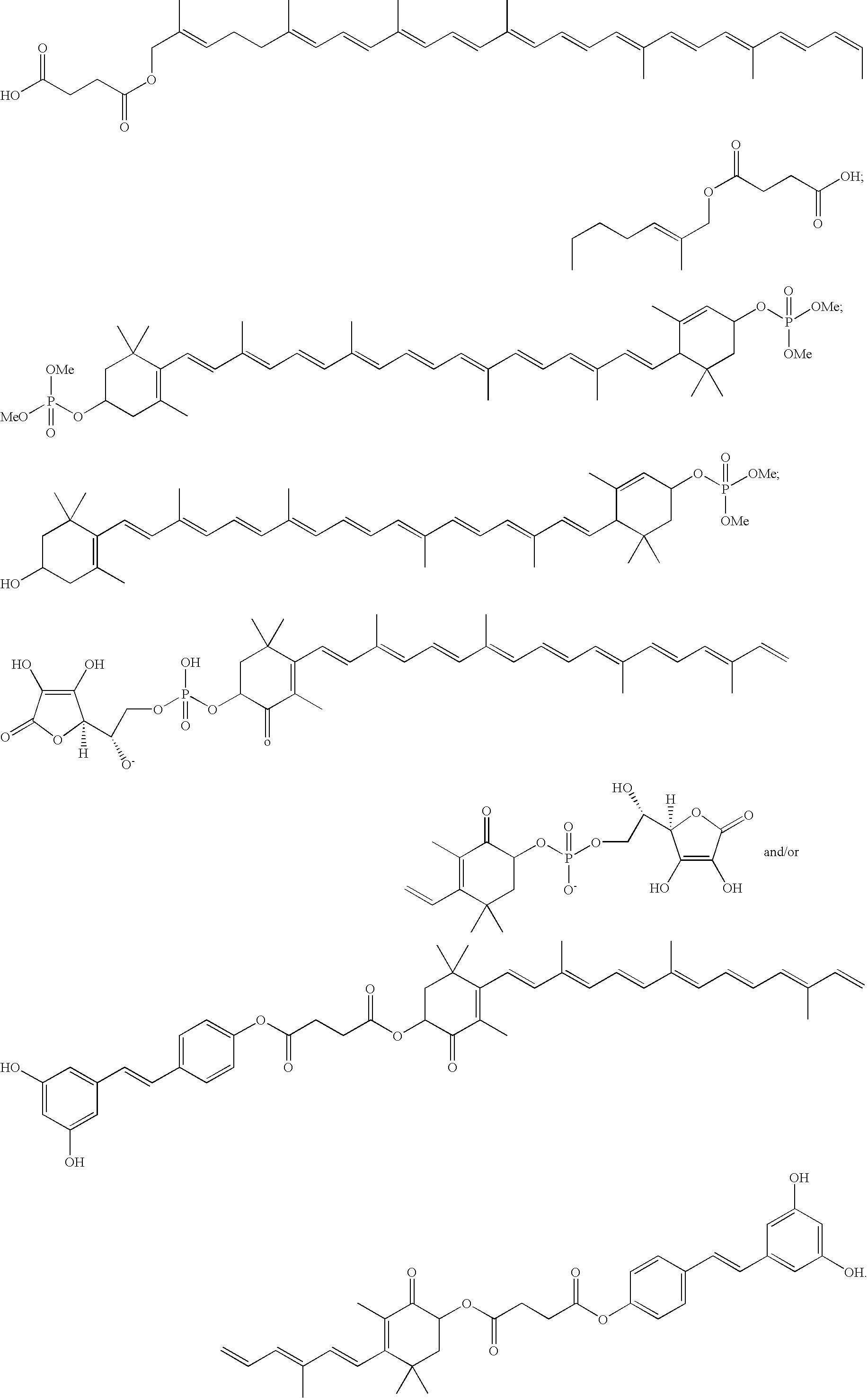

Further details regarding the synthesis of carotenoid derivatives and analogs is illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and PCT International Application Number PCT/US2003/023706 filed on Jul. 29, 2003, entitled “STRUCTURAL CAROTENOID ANALOGS FOR THE INHIBITION AND AMELIORATION OF DISEASE” by Lockwood et al. (International Publication Number WO 2004/011423 A2, published on Feb. 5, 2004) both of which are incorporated by reference as though fully set forth herein.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/mL. In certain embodiments, water-soluble carotenoid analogs or derivatives may have a water solubility of greater than about 10 mg/mL. In certain embodiments, water-soluble carotenoid analogs or derivatives may have a water solubility of greater than about 20 mg/mL. In some embodiments, water-soluble carotenoid analogs or derivatives may have a water solubility of greater than about 50 mg/mL.

Naturally occurring carotenoids such as xanthophyll carotenoids of the C40 series, which includes commercially important compounds such as lutein, zeaxanthin, and astaxanthin, have poor aqueous solubility in the native state. Varying the chemical structure(s) of the esterified moieties may vastly increase the aqueous solubility and/or dispersibility of derivatized carotenoids.

In some embodiments, highly water-dispersible C40 carotenoid derivatives may include natural source RRR-lutein (β,ε-carotene-3,3′-diol) derivatives. Derivatives may be synthesized by esterification with inorganic phosphate and succinic acid, respectively, and subsequently converted to the sodium salts. Deep orange, evenly colored aqueous suspensions were obtained after addition of these derivatives to USP-purified water. Aqueous dispersibility of the disuccinate sodium salt of natural lutein was 2.85 mg/mL; the diphosphate salt demonstrated a >10-fold increase in dispersibility at 29.27 mg/mL. Aqueous suspensions may be obtained without the addition of heat, detergents, co-solvents, or other additives.

The direct aqueous superoxide scavenging abilities of these derivatives were subsequently evaluated by electron paramagnetic resonance (EPR) spectroscopy in a well-characterized in vitro isolated human neutrophil assay. The derivatives may be potent (millimolar concentration) and nearly identical aqueous-phase scavengers, demonstrating dose-dependent suppression of the superoxide anion signal (as detected by spin-trap adducts of DEPMPO) in the millimolar range. Evidence of card-pack aggregation was obtained for the diphosphate derivative with UV-V is spectroscopy (discussed herein), whereas limited card-pack and/or head-to-tail aggregation was noted for the disuccinate derivative. These lutein-based soft drugs may find utility in those commercial and clinical applications for which aqueous-phase singlet oxygen quenching and direct radical scavenging may be required.

The absolute size of a carotenoid derivative (in 3 dimensions) is important when considering its use in biological and/or medicinal applications. Some of the largest naturally occurring carotenoids are no greater than about C₅₀. This is probably due to size limits imposed on molecules requiring incorporation into and/or interaction with cellular membranes. Cellular membranes may be particularly co-evolved with molecules of a length of approximately 30 nm. In some embodiments, carotenoid derivatives may be greater than or less than about 30 nm in size. In certain embodiments, carotenoid derivatives may be able to change conformation and/or otherwise assume an appropriate shape, which effectively enables the carotenoid derivative to efficiently interact with a cellular membrane.

Although the above structure, and subsequent structures, depict alkenes in the E configuration this should not be seen as limiting. Compounds discussed herein may include embodiments where alkenes are in the Z configuration or include alkenes in a combination of Z and E configurations within the same molecule. The compounds depicted herein may naturally convert between the Z and E configuration and/or exist in equilibrium between the two configurations.

Compounds described herein embrace isomers mixtures, racemic, optically active, and optically inactive stereoisomers and compounds. Carotenoid analogs or derivatives may have increased water solubility and/or water dispersibility relative to some or all known naturally occurring carotenoids. In some embodiments, one or more co-antioxidants may be coupled to a carotenoid or carotenoid derivative or analog.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate, succinate, co-antioxidant (e.g., Vitamin C, Vitamin C analogs, Vitamin C derivatives, Vitamin E, Vitamin E analogs, Vitamin E derivatives, or flavonoids), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

The synthesis of water-soluble and/or water-dispersible carotenoids (e.g., C40) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their bioavailability and hence proper evaluation of efficacy. Esterification or etherification may be useful to increase oral bioavailability, a fortuitous side effect of the esterification process, which can increase solubility in gastric mixed micelles. These compounds, upon introduction to the mammalian GI tract, are rapidly and effectively cleaved to the parent, non-esterified compounds, and enter the systemic circulation in that manner and form. The effect of the intact ester and/or ether compound on the therapeutic endpoint of interest can be obtained with parenteral administration of the compound(s). The net overall effect is an improvement in potential clinical utility for the lipophilic carotenoid compounds as therapeutic agents.

In one embodiment, a human subject may be administered a pharmaceutical composition comprising a carotenoid analog or derivative. The analog or derivative may be broken down according to the following reaction:

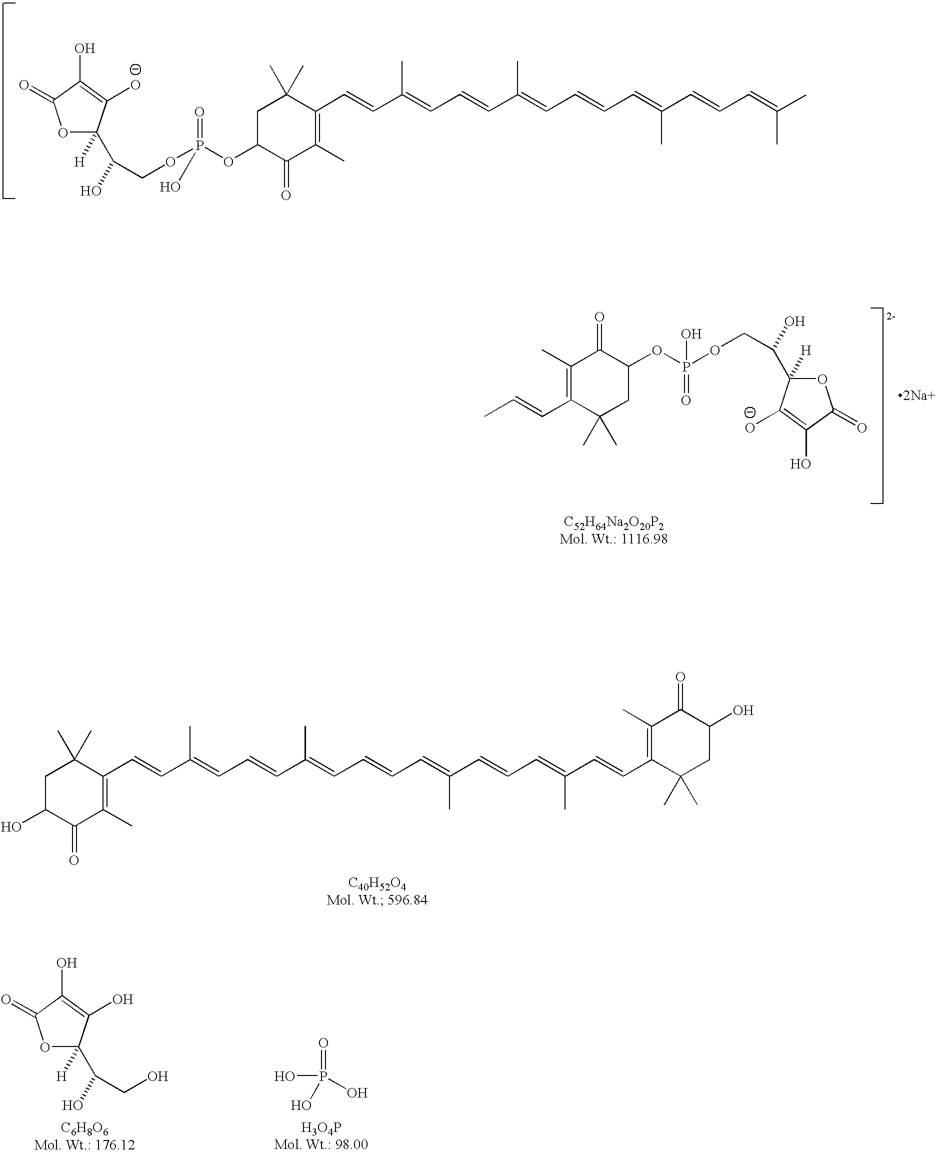

In some embodiments, the principles of retrometabolic drug design may be utilized to produce novel soft drugs from the asymmetric parent carotenoid scaffold (e.g., RRR-lutein (β,ε-carotene-3,3′-diol)). For example, lutein scaffold for derivatization was obtained commercially as purified natural plant source material, and was primarily the RRR-stereoisomer (one of 8 potential stereoisomers). Lutein (Scheme 1) possesses key characteristics—similar to starting material astaxanthin—which make it an ideal starting platform for retrometabolic syntheses: (1) synthetic handles (hydroxyl groups) for conjugation, and (2) an excellent safety profile for the parent compound. As stated above, lutein is available commercially from multiple sources in bulk as primarily the RRR-stereoisomer, the primary isomer in the human diet and human retinal tissue.

In some embodiments, carotenoid analogs or derivatives may have increased water solubility and/or water dispersibility relative to some or all known naturally occurring carotenoids.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a human subject or patient.

Some embodiments may include solutions or pharmaceutical preparations of carotenoids and/or carotenoid derivatives combined with co-antioxidants, in particular vitamin C and/or vitamin C analogs or derivatives. Pharmaceutical preparations may include about a 2:1 ratio of vitamin C to carotenoid respectively.

In some embodiments, co-antioxidants (e.g., vitamin C) may increase solubility of the chemical compound. In certain embodiments, co-antioxidants (e.g., vitamin C) may decrease toxicity associated with at least some carotenoid analogs or derivatives. In certain embodiments, co-antioxidants (e.g., vitamin C) may increase the potency of the chemical compound synergistically. Co-antioxidants may be coupled (e.g., a covalent bond) to the carotenoid derivative. Co-antioxidants may be included as a part of a pharmaceutically acceptable formulation.

As used herein the terms “structural carotenoid analogs or derivatives” may be generally defined as carotenoids and the biologically active structural analogs or derivatives thereof. “Derivative” in the context of this application is generally defined as a chemical substance derived from another substance either directly or by modification or partial substitution. “Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Parent carotenoids are selected from the more than 700 naturally occurring carotenoids described in the literature, and their stereo- and geometric isomers. Such analogs or derivatives may include, but are not limited to, esters, ethers, carbonates, amides, carbamates, phosphate esters and ethers, sulfates, glycoside ethers, with or without spacers (linkers).

As used herein the terms “the synergistic combination of more than one xanthophyll carotenoid or structural analog or derivative or synthetic intermediate of carotenoids” may be generally defined as any composition including one xanthophyll carotenoid or a structural carotenoid analog or derivative or synthetic intermediate combined with one or more different xanthophyll carotenoids or structural carotenoid analogs or derivatives or synthetic intermediates or co-antioxidants, either as derivatives or in solutions and/or formulations.

Certain embodiments may include administering a xanthophyll carotenoid or a structural carotenoid analogs or derivatives or synthetic intermediates alone or in combination to a human subject such that at least a portion of the adverse effects of elevated systemic and/or target organ oxidative stress are thereby reduced, inhibited and/or ameliorated. The xanthophyll carotenoid or a structural carotenoid analogs or derivatives or synthetic intermediates may be water-soluble and/or water dispersible derivatives. The carotenoid derivatives may include any substituent that substantially increases the water solubility of the naturally occurring carotenoid. The carotenoid derivatives may retain and/or improve the antioxidant properties of the parent carotenoid. The carotenoid derivatives may retain the non-toxic properties of the parent carotenoid. The carotenoid derivatives may have increased bioavailability, relative to the parent carotenoid, upon administration to a human subject. The parent carotenoid may be naturally occurring.

Other embodiments may include the administering a composition comprised of the synergistic combination of more than one xanthophyll carotenoids or structural carotenoid analogs or derivatives or synthetic intermediates to a human subject such that the systemic oxidative stress experienced the body or a body organ of a human subject is thereby reduced, inhibited and/or ameliorated. The composition may be a “racemic” (i.e. mixture of the potential stereoisomeric forms) mixture of carotenoid derivatives. Included as well are pharmaceutical compositions comprised of structural analogs or derivatives or synthetic intermediates of carotenoids in combination with a pharmaceutically acceptable carrier. In one embodiment, a pharmaceutically acceptable carrier may be serum albumin. In one embodiment, structural analogs or derivatives or synthetic intermediates of carotenoids may be complexed with human serum protein such as, for example, human serum albumin (i.e., HSA) in a solvent. In an embodiment, HSA may act as a pharmaceutically acceptable carrier.

In some embodiments, a single stereoisomer of a structural analog or derivative or synthetic intermediate of carotenoids may be administered to a human subject in order to ameliorate a pathological condition. Administering a single stereoisomer of a particular compound (e.g., as part of a pharmaceutical composition) to a human subject may be advantageous (e.g., increasing the potency of the pharmaceutical composition). Administering a single stereoisomer may be advantageous due to the fact that only one isomer of potentially many may be biologically active enough to have the desired effect.

In some embodiments, compounds described herein may be administered in the form of nutraceuticals. “Nutraceuticals” as used herein, generally refers to dietary supplements, foods, or medical foods that: 1. possess health benefits generally defined as reducing the risk of a disease or health condition, including the management of a disease or health condition or the improvement of health; and 2. are safe for human consumption in such quantity, and with such frequency, as required to realize such properties. Generally a nutraceutical is any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages. It is important to note that this definition applies to all categories of food and parts of food, ranging from dietary supplements such as folic acid, used for the prevention of spina bifida, to chicken soup, taken to lessen the discomfort of the common cold. This definition also includes a bio-engineered designer vegetable food, rich in antioxidant ingredients, and a stimulant functional food or pharmafood. Within the context of the description herein where the composition, use and/or delivery of pharmaceuticals are described nutraceuticals may also be composed, used, and/or delivered in a similar manner where appropriate.

Dosage and Administration

The xanthophyll carotenoid, carotenoid derivative or analog may be administered at a dosage level up to conventional dosage levels for xanthophyll carotenoids, carotenoid derivatives or analogs, but will typically be less than about 2 gm per day. Suitable dosage levels may depend upon the overall systemic effect of the chosen xanthophyll carotenoids, carotenoid derivatives or analogs, but typically suitable levels will be about 0.001 to 50 mg/kg body weight of the patient per day, from about 0.005 to 30 mg/kg per day, or from about 0.05 to 10 mg/kg per day. The compound may be administered on a regimen of up to 6 times per day, between about 1 to 4 times per day, or once per day.

In the case where an oral composition is employed, a suitable dosage range is, e.g. from about 0.01 mg to about 100 mg of a xanthophyll carotenoid, carotenoid derivative or analog per kg of body weight per day, preferably from about 0.1 mg to about 10 mg per kg and for cytoprotective use from 0.1 mg to about 100 mg of a xanthophyll carotenoid, carotenoid derivative or analog per kg of body weight per day.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

In some embodiments, compositions may include all compositions of 1.0 gram or less of a particular structural carotenoid analog, in combination with 1.0 gram or less of one or more other structural carotenoid analogs or derivatives or synthetic intermediates and/or co-antioxidants, in an amount which is effective to achieve its intended purpose. While individual subject needs vary, determination of optimal ranges of effective amounts of each component is with the skill of the art. Typically, a structural carotenoid analog or derivative or synthetic intermediates may be administered to mammals, in particular humans, orally at a dose of 5 to 100 mg per day referenced to the body weight of the mammal or human being treated for a particular disease. Typically, a structural carotenoid analog or derivative or synthetic intermediate may be administered to mammals, in particular humans, parenterally at a dose of between 5 to 1000 mg per day referenced to the body weight of the mammal or human being treated for a particular disease. In other embodiments, about 100 mg of a structural carotenoid analog or derivative or synthetic intermediate is either orally or parenterally administered to treat or prevent disease.

The unit oral dose may comprise from about 0.25 mg to about 1.0 gram, or about 5 to 25 mg, of a structural carotenoid analog. The unit parenteral dose may include from about 25 mg to 1.0 gram, or between 25 mg and 500 mg, of a structural carotenoid analog. The unit intracoronary dose may include from about 25 mg to 1.0 gram, or between 25 mg and 100 mg, of a structural carotenoid analog. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a structural carotenoid analog or derivative or synthetic intermediate as a raw chemical, the compounds may be administered as part of a pharmaceutical preparation containing suitable pharmaceutically acceptable carriers, preservatives, excipients and auxiliaries which facilitate processing of the structural carotenoid analog or derivative or synthetic intermediates which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

General guidance in determining effective dose ranges for pharmacologically active compounds and compositions for use in the presently described embodiments may be found, for example, in the publications of the International Conference on Harmonisation and in REMINGTON'S PHARMACEUTICAL SCIENCES, 8^{th} Edition Ed. Bertram G. Katzung, chapters 27 and 28, pp. 484-528 (Mack Publishing Company 1990) and yet further in BASIC & CLINICAL PHARMACOLOGY, chapters 5 and 66, (Lange Medical Books/McGraw-Hill, New York, 2001).

Pharmaceutical Compositions

Any suitable route of administration may be employed for providing a patient with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any methods well known in the pharmaceutical arts.

For administration by inhalation, the drugs used in the present invention are conveniently delivered in the form of an aerosol spray presentation from pressurized packs or nebulisers. The compounds may also be delivered as powders, which may be formulated, and the powder composition may be inhaled with the aid of an insufflation powder inhaler device.

Suitable topical formulations for use in the present embodiments may include transdermal devices, aerosols, creams, ointments, lotions, dusting powders, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the structural carotenoid analog. Liposomal formulations, in which mixtures of the structural carotenoid analog or derivative with, for example, egg yolk phosphotidylcholine (E-PC), may be made for injection. Optionally, the suspension may contain stabilizers, for example, antioxidants such as BHT, and/or preservatives, such as benzyl alcohol.

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to systemically inhibit or reduce oxidative stress experienced by the body or body organ of a human subject, or for modulating the redox status of a body organ.

By way of general guidance, the daily oral dosage of each active ingredient, when used for the indicated effects, will range between about 0.001 to 1000 mg/kg of body weight, between about 0.01 to 100 mg/kg of body weight per day, or between about 1.0 to 20 mg/kg/day. Intravenously administered doses may range from about 1 to about 10 mg/kg/minute during a constant rate infusion. Compounds of this invention may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams or more of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

### EXAMPLES

Having now described the invention, the same will be more readily understood through reference to the following example(s), which are provided by way of illustration, and are not intended to be limiting of the present invention.

General. Natural source lutein (90%) was obtained from ChemPacific, Inc. (Baltimore, Md.) as a red-orange solid and was used without further purification. All other reagents and solvents used were purchased from Acros (New Jersey, USA) and were used without further purification. All reactions were performed under N₂ atmosphere. All flash chromatographic purifications were performed on Natland International Corporation 230-400 mesh silica gel using the indicated solvents. LC/MS (APCI) and LC/MS (ESI) were recorded on an Agilent 1100 LC/MSD VL system; column: Zorbax Eclipse XDB-C18 Rapid Resolution (4.6×75 mm, 3.5 μm, USUT002736); temperature: 25° C.; starting pressure: 105 bar; flow rate: 1.0 mL/min; mobile phase (% A=0.025% trifluoroacetic acid in H₂O, % B=0.025% trifluoroacetic acid in acetonitrile) Gradient program: 70% A/30% B (start), step gradient to 50% B over 5 min, step gradient to 98% B over 8.30 min, hold at 98% B over 25.20 min, step gradient to 30% B over 25.40 min; PDA Detector: 470 nm. The presence of trifluoroacetic acid in the LC eluents acts to protonate synthesized lutein disuccinate and diphosphate salts to give the free di-acid forms, yielding M⁺=768 for the disuccinate salt sample and M⁺=728 for the diphosphate salt sample in MS analyses. LRMS: +mode; ESI: electrospray chemical ionization, ion collection using quadrapole; APCI: atmospheric pressure chemical ionization, ion collection using quadrapole. MS (ESI-IT) was recorded on a HCT plus Bruker Daltonics Mass Spectrometer system, LRMS: +mode; ESI-IT: electrospray chemical ionization, ion collection using ion trap. ¹H NMR analyses were attempted on Varian spectrometers (300 and 500 MHz). NMR analyses of natural source lutein as well as synthesized lutein derivatives yielded only partially discernable spectra, perhaps due to the presence of interfering impurities (natural source lutein), or due to aggregation (natural source lutein and derivatives). In attempts to circumvent the problems associated with NMR analyses, samples were prepared using mixtures of deuterated solvents including methanol/chloroform, methanol/water, methyl sulfoxide/water, and chloroform/methanol/water. However, such attempts failed to give useful data.

Natural source lutein (β,ε-carotene-3,3′-diol), 1. LC/MS (ESI): 9.95 min (2.78%), λₘₐₓ 226 nm (17%), 425 nm (100%); 10.58 min (3.03%), λₘₐₓ 225 nm (21%), 400 nm (100%); 11.10 min (4.17%), λₘₐₓ 225 nm (16%), 447 nm (100%); 12.41 min (90.02%), λₘₐₓ 269 nm (14%), 447 nm (100%), m/z 568 M⁺ (69%), 551 [M-H₂O+H]⁺ (100%), 533 [M-2H₂O+H]⁺ (8%).

β,ε-carotenyl 3,3′-disuccinate, 2. To a solution of natural source lutein (1) (0.50 g, 0.879 mmol) in CH₂Cl₂ (8 mL) was added N,N-diisopropylethylamine (3.1 mL, 17.58 mmol) and succinic anhydride (0.88 g, 8.79 mmol). The solution was stirred at RT overnight and then diluted with CH₂Cl₂ and quenched with water/1 M HCl (5/1). The aqueous layer was extracted two times with CH₂Cl₂ and the combined organic layer washed three times with cold water/1 M HCl (5/1), dried over Na₂SO₄, and concentrated. The resulting red-orange oil washed (slurried) three times with hexanes to yield disuccinate 2 (0.433 g, 64%) as a red-orange solid; LC/MS (APCI): 10.37 min (4.42%), λₘₐₓ 227 nm (56%), 448 nm (100%), m/z 769 [M+H]⁺ (8%), 668 [M-C₄O₃H₄]⁺ (9%), 637 (36%), 138 (100%); 11.50 min (92.40%), λₘₐₓ 269 nm (18%), 447 nm (100%), m/z 769 [M+H]⁺ (7%), 668 [M-C₄O₃H₄]⁺ (9%), 651 (100%); 12.03 min (3.18%) 227 nm (55%), 446 nm (100%), m/z 668 [M-C₄O₃H₄]⁺ (15%), 550 (10%), 138 (100%)

β,ε-carotenyl 3,3′-disuccinate sodium salt, 3. To a solution of disuccinate 2 (0.32 g, 0.416 mmol) in CH₂Cl₂/methanol (5 mL/1 mL) at 0° C. was added drop-wise sodium methoxide (25% wt in methanol; 0.170 mL, 0.748 mmol). The solution was stirred at RT overnight and then quenched with water and stirred for 5 min. The solution was then concentrated and the aqueous layer washed four times with Et₂O. Lyophilization of the clear, red-orange aqueous solution yielded 3 (0.278 g, 91%) as an orange, hygroscopic solid; LC/MS (APCI): 11.71 min (94.29%), λₘₐₓ 269 nm (18%), 446 nm (100%), m/z 769 [M-2Na+3H]⁺ (8%), 668 [M-2Na+2H—C₄O₃H₄]⁺ (6%), 651 (100%); 12.74 min (5.71%), λₘₐₓ 227 nm (30%), 269 nm (18%), 332 nm (39%), 444 nm (100%), m/z 768 [M-2Na+2H]⁺ (2%), 668 [M-2Na+2H—C₄O₃H₄]⁺ (3%), 651 (12%), 138 (100%)

Tribenzyl phosphite, 4. To a well-stirred solution of phosphorus trichloride (1.7 mL, 19.4 mmol) in Et₂O (430 mL) at 0° C. was added dropwise a solution of triethylamine (8.4 mL, 60.3 mmol) in Et₂O (20 mL), followed by a solution of benzyl alcohol (8.1 mL, 77.8 mmol) in Et₂O (20 mL). The mixture was stirred at 0° C. for 30 min and then at RT overnight. The mixture was filtered and the filtrate concentrated to give a colorless oil. Silica chromatography (hexanes/Et₂O/triethylamine, 4/1/1%) of the crude product yielded 4 (5.68 g, 83%) as a clear, colorless oil that was stored under N₂ at −20° C.; ¹H NMR: δ 7.38 (15H, m), 4.90 (6H, d)

Dibenzyl phosphoroiodidate, 5. To a solution of tribenzyl phosphite (5.43 g, 15.4 mmol) in CH₂Cl₂ (8 mL) at 0° C. was added 12 (3.76 g, 14.8 mmol). The mixture was stirred at 0° C. for 10 min or until the solution became clear and colorless. The solution was then stirred at RT for 10 min and used directly in the next step.

3-(Bis benzyl-phosphoryloxy)-3′-(phosphoryloxy)-β,ε-carotene, 6. To a solution of natural source lutein (1) (0.842 g, 1.48 mmol) in CH₂Cl₂ (8 mL) was added pyridine (4.8 mL, 59.2 mmol). The solution was stirred at 0° C. for 5 min and then freshly prepared 5 (14.8 mmol) in CH₂Cl₂ (8 mL) was added drop-wise to the mixture at 0° C. The solution was stirred at 0° C. for 1 h and then diluted with CH₂Cl₂ and quenched with brine. The aqueous layer was extracted twice with CH₂Cl₂ and the combined organic layer washed once with brine, then dried over Na₂SO₄ and concentrated. Pyridine was removed from the crude red oil by azeotropic distillation using toluene. The crude product was alternately washed (slurried) twice with hexanes and Et₂O to yield 6 as a red oil, used in the next step without further purification; LC/MS (ESI): 9.93 min (44.78%), λₘₐₓ 267 nm (33%), 444 nm (100%), m/z 890 [M-H₂O]⁺ (8%), 811 [M-PO₃H—H₂O+H]⁺ (73%), 533 (100%); 9.99 min (29.0%), λₘₐₓ 268 nm (24%), 446 nm (100%), m/z 890 [M-H₂O]⁺ (6%), 811 [M-PO₃H—H₂O+H]⁺ (72%), 533 (100%); 10.06 min (26.23%), L 266 nm (15%), 332 nm (22%), 444 nm (100%), m/z 890 [M-H₂O]⁺ (5%), 811 [M-PO₃H—H₂O+H]⁺ (90%), 533 (100%)

3-(Bis benzyl-phosphoryloxy)-3′-hydroxy-β,ε-carotene, 7. To a solution of 6 (0.033 mmol) in tetrahydrofuran/water (1 mL/0.5 mL) at 0° C. was added LiOH—H₂O (0.003 g, 0.073 mmol). The solution was stirred at RT for 1 h and then quenched with methanol. The crude reaction mixture was analyzed by LC/MS; LC/MS (ESI): 10.02 min (40.60%), λₘₐₓ 266 nm (12%), 333 nm (25%), 445 nm (100%), m/z 890 [M-H₂O]⁺ (33%), 811 [M-PO₃H—H₂O+H]⁺ (50%), 533 (100%); 16.37 min (49.56%) λₘₐₓ 267 nm (16%), 332 nm (27%), 446 nm (100%), m/z 828 M⁺ (55%), 550 (44%)

3,3′-Diphosphoryloxy-β,ε-carotene, 8. To a solution of 6 (1.48 mmol) in CH₂Cl₂ (10 mL) at 0° C. was added drop-wise N,O-bis(trimethylsilyl)acetamide (3.7 mL, 14.8 mmol) and then bromotrimethylsilane (1.56 mL, 11.8 mmol). The solution was stirred at 0° C. for 1 h, quenched with methanol, diluted with CH₂Cl₂, and then concentrated. The resulting red oil was alternately washed (slurried) three times with ethyl acetate and CH₂Cl₂ to yield crude phosphate 8 (2.23 g) as a dark orange oil, used in the next step without further purification; LC/MS (ESI): 8.55 min (45.67%), λₘₐₓ 214 nm (25%), 268 nm (28%), 447 nm (100%), m/z 631 [M-PO₃H—H₂O+H]⁺ (30%), 533 (18%), 279 (13%), 138 (87%); 8.95 min (35.0%), λₘₐₓ 217 nm (14%), 268 nm (23%), 448 nm (100%), m/z 631 [M-PO₃H—H₂O+H]⁺ (26%), 533 (32%), 279 (18%), 138 (100%); 9.41 min (9.70%), w 225 nm (37%), 269 nm (23%), 335 nm (19%), 447 nm (100%), m/z 631 [M-PO₃H—H₂O+H]⁺ (6%), 533 (18%), 279 (13%), 138 (100%)

3,3′-Diphosphoryloxy-β,ε-carotene sodium salt, 9. To a solution of crude 8 (ca 50%; 2.23 g, 3.06 mmol) in methanol (20 mL) at 0° C. was added drop-wise sodium methoxide (25%; 3.5 mL, 15.3 mmol). The solution was stirred at RT for 2 h and the resulting orange solid washed (slurried) three times with methanol. Water was added to the moist solid and the resulting aqueous layer was extracted with CH₂Cl₂, ethyl acetate, and again with CH₂Cl₂. Lyophilization of the clear, red-orange aqueous solution yielded 9 (0.956 g, 80% over 3 steps) as an orange, hygroscopic solid; LC/MS (ESI): 7.81 min (22.34%), λₘₐₓ 215 nm (34%), 268 nm (30%), 448 nm (100%), m/z 711 [M-4Na—H₂O+SH]⁺ (9%), 533 (13%), 306 (100%); 8.33 min (39.56%), λₘₐₓ 217 nm (14%), 268 nm (20%), 448 nm (100%), m/z 711 [M-4Na—H₂O+5H]⁺ (10%), 533 (11%), 306 (100%); 8.90 min (38.09%), λₘₐₓ 223 nm (45%), 269 nm (30%), 336 nm (26%), 448 nm (100%), m/z 711 [M-4Na—H₂O+5H]⁺ (8%), 631 [M-4Na—PO₃H—H₂O+5H]⁺ (18%), 533 (20%), 306 (100%); MS (ESI-IT): m/z 816 M⁺ (55%), 772 [M-2Na+2H]⁺ (37%), 728 [M-4Na+4H]⁺ (74%)

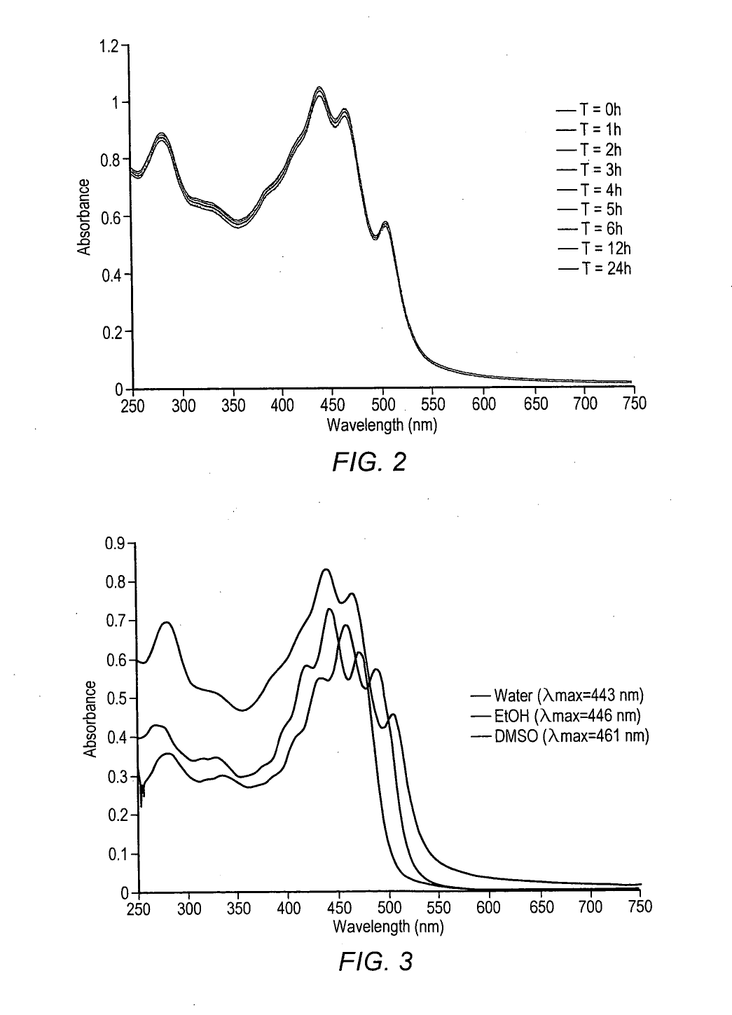

UV/Visible spectroscopy. For spectroscopic sample preparations, 3 and 9 were dissolved in the appropriate solvent to yield final concentrations of approximately 0.01 mM and 0.2 mM, respectively. The solutions were then added to a rectangular cuvette with 1 cm path length fitted with a glass stopper. The absorption spectrum was subsequently registered between 250 and 750 nm. All spectra were accumulated one time with a bandwidth of 1.0 nm at a scan speed of 370 nm/min. For the aggregation time-series measurements, spectra were obtained at baseline (immediately after solvation; time zero) and then at the same intervals up to and including 24 hours post-solvation (see FIG. 2-FIG. 7). Concentration was held constant in the ethanolic titration of the diphosphate lutein sodium salt, for which evidence of card-pack aggregation was obtained (FIG. 5-FIG. 7).

Determination of aqueous solubility/dispersibility. 30.13 mg of 3 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/V is spectroscopy at 436 nm, and the absorbance was compared to a standard curve compiled from 4 standards of known concentration. The concentration of the original supernatant was calculated to be 2.85 mg/mL and the absorptivity was 36.94 AU*mL/cm*mg. Slight error may have been introduced by the small size of the original aliquot.

Next, 30.80 mg of 9 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/Vis spectroscopy at 411 nm, and the absorbance was compared to a standard curve compiled from 4 standards of known concentration. The concentration of the original supernatant was calculated to be 29.27 mg/mL and the absorptivity was 2.90 AU*mL/cm*mg. Slight error may have been introduced by the small size of the original aliquot.

Leukocyte Isolation and Preparation. Human polymorphonuclear leukocytes (PMNs) were isolated from freshly sampled venous blood of a single volunteer (S.F.L.) by Percoll density gradient centrifugation as described previously. Briefly, each 10 mL of whole blood was mixed with 0.8 mL of 0.1 M EDTA and 25 mL of saline. The diluted blood was then layered over 9 mL of Percoll at a specific density of 1.080 g/mL. After centrifugation at 400×g for 20 min at 20° C., the plasma, mononuclear cell, and Percoll layers were removed. Erythrocytes were subsequently lysed by addition of 18 mL of ice-cold water for 30 s, followed by 2 mL of 10×PIPES buffer (25 mM PIPES, 110 mM NaCl, and 5 mM KCl, titrated to pH 7.4 with NaOH). Cells were then pelleted at 4° C., the supernatant was decanted, and the procedure was repeated. After the second hypotonic cell lysis, cells were washed twice with PAG buffer [PIPES buffer containing 0.003% human serum albumin (HSA) and 0.1% glucose]. Afterward, PMNs were counted by light microscopy on a hemocytometer. The isolation yielded PMNs with a purity of >95%. The final pellet was then suspended in PAG-CM buffer (PAG buffer with 1 mM CaCl₂ and 1 mM MgCl₂).

EPR Measurements. All EPR measurements were performed using a Bruker ER 300 EPR spectrometer operating at X-band with a TM₁₁₀ cavity as previously described. The microwave frequency was measured with a Model 575 microwave counter (EIP Microwave, Inc., San Jose, Calif.). To measure superoxide anion (O⁻₂) generation from phorbol-ester (PMA)-stimulated PMNs, EPR spin-trapping studies were performed using the spin trap DEPMPO (Oxis, Portland, Oreg.) at 10 mM. 1×10⁶ PMNs were stimulated with PMA (1 ng/mL) and loaded into capillary tubes for EPR measurements. To determine the radical scavenging ability of 3 and 9 in aqueous and ethanolic formulations, PMNs were pre-incubated for 5 minutes with test compound, followed by PMA stimulation.

Instrument settings used in the spin-trapping experiments were as follows: modulation amplitude, 0.32 G; time constant, 0.16 s; scan time, 60 s; modulation frequency, 100 kHz; microwave power, 20 milliwatts; and microwave frequency, 9.76 GHz. The samples were placed in a quartz EPR flat cell, and spectra were recorded. The component signals in the spectra were identified and quantified as reported previously.

UV/Vis Spectral Properties in Organic and Aqueous Solvents.

UV-Vis spectral evaluation of the disuccinate lutein sodium salt is depicted in FIG. 2-FIG. 4. FIG. 2 depicts a time series of the UV/Vis absorption spectra of the disodium disuccinate derivative of natural source lutein in water. The 4 (443 nm) obtained at time zero did not appreciably blue-shift over the course of 24 hours, vibrational fine structure was maintained (% III/II=35%), and the spectra became only slightly hypochromic (i.e. decreased in absorbance intensity) over time, indicating minimal time-dependent supramolecular assembly (aggregation) of the card-pack type during this time period. Existence of head-to-tail (J-type) aggregation in solution cannot be ruled out.

FIG. 3 depicts a UV/Vis absorption spectra of the disodium disuccinate derivative of natural source lutein in water (λₘₐₓ=443 nm), ethanol (4=446 nm), and DMSO (λₘₐₓ=461 nm). Spectra were obtained at time zero. A prominent cis peak is seen with a maximum at 282 nm in water. The expected bathochromic shift of the spectrum in the more polarizable solvent (DMSO) is seen (461 nm). Only a slight hypsochromic shift is seen between the spectrum in water and that in ethanol, reflecting minimal card-pack aggregation in aqueous solution. Replacement of the main visible absorption band observed in EtOH by an intense peak in the near UV region—narrow and displaying no vibrational fine structure—is not observed in the aqueous solution of this highly water-dispersible derivative, in comparison to the spectrum of pure lutein in an organic/water mixture.

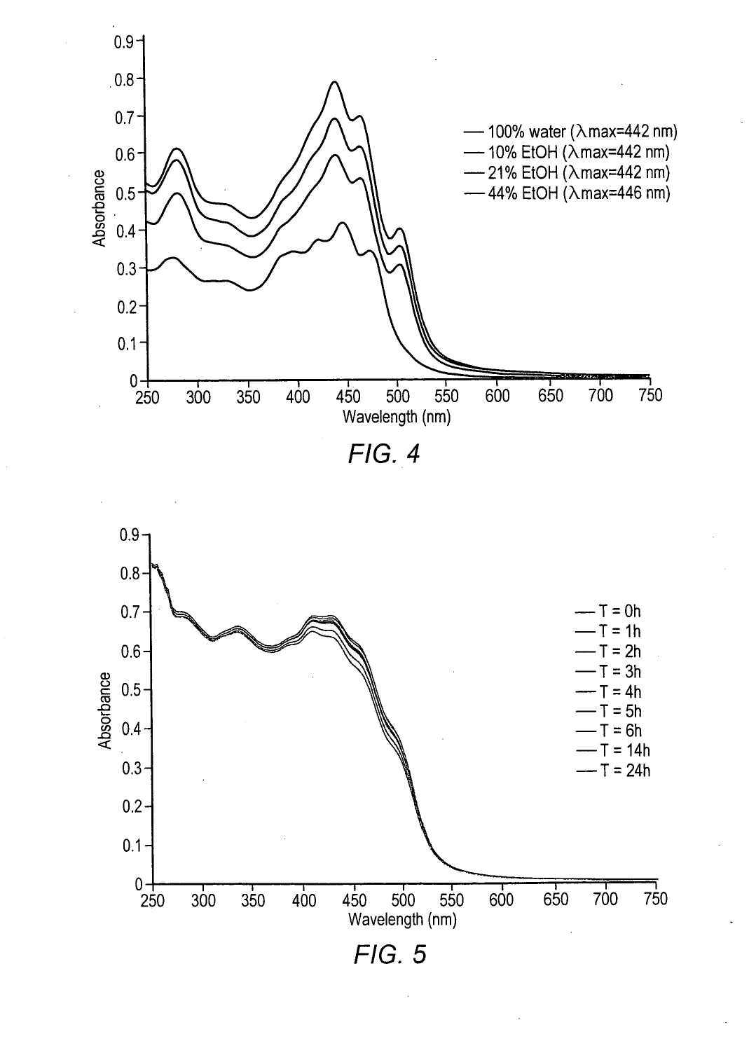

FIG. 4 depicts a UV/Vis absorption spectra of the disodium disuccinate derivative of natural source lutein in water (λₘₐₓ=442 nm) with increasing concentrations of ethanol. The 4 increases to 446 nm at an EtOH concentration of 44%, at which point no further shift of the absorption maximum occurs (i.e. a molecular solution has been achieved), identical to that obtained in 100% EtOH (See FIG. 3).

UV-Vis spectral evaluation of the diphosphate lutein sodium salt is depicted in FIG. 5-FIG. 7. FIG. 5 depicts a time series of the U/V is absorption spectra of the disodium diphosphate derivative of natural source lutein in water. Loss of vibrational fine structure (spectral distribution beginning to approach unimodality) and the blue-shifted lambda max relative to the lutein chromophore in EtOH suggested that card-pack aggregation was present immediately upon solvation. The λₘₐₓ (428 nm) obtained at time zero did not appreciably blue-shift over the course of 24 hours, and the spectra became slightly more hypochromic over time (i.e. decreased in absorbance intensity), indicating additional time-dependent supramolecular assembly (aggregation) of the card-pack type during this time period. This spectrum was essentially maintained over the course of 24 hours (compare with FIG. 2, disuccinate lutein sodium salt).

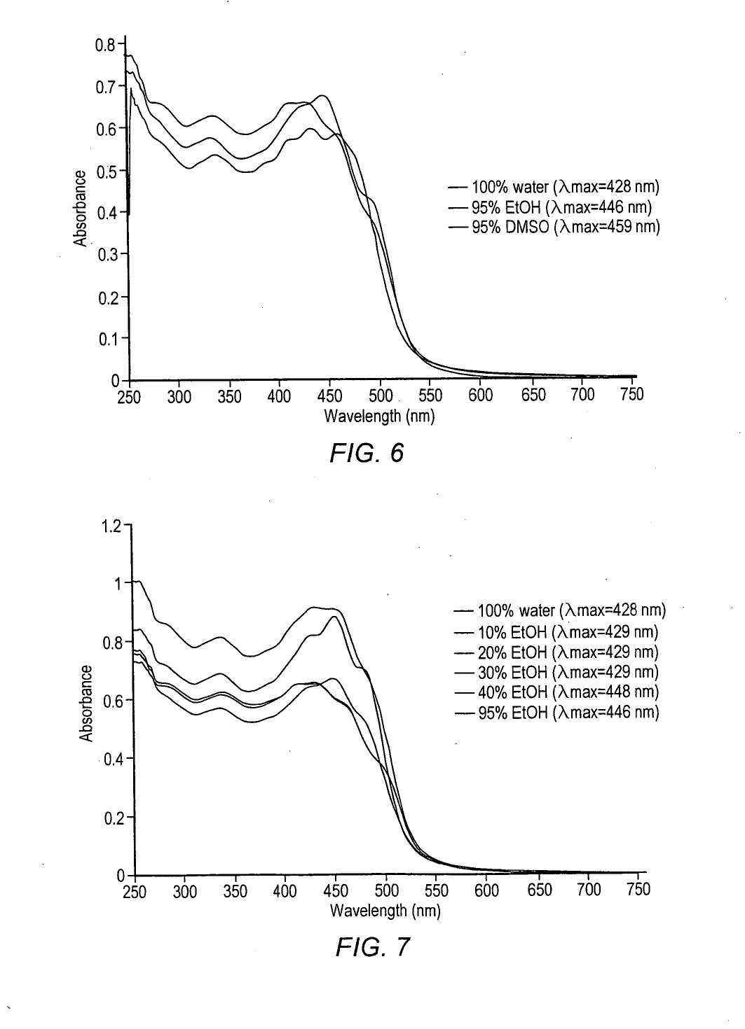

FIG. 6 depicts a UV/Vis absorption spectra of the disodium diphosphate derivative of natural source lutein in 95% ethanol (λₘₐₓ=446 nm), 95% DMSO (λₘₐₓ=459 nm), and water (4=428 nm n). A red-shift was observed (4 to 446 nm), as was observed with the disuccinate derivate. Wetting of the diphosphate lutein derivative with a small amount of water was required to obtain appreciable solubility in organic solvent (e.g. EtOH and DMSO). Spectra were obtained at time zero. The expected bathochromic shift (in this case to 459 nm) of the spectrum in the more polarizable solvent (95% DMSO) is seen. Increased vibrational fine structure and red-shifting of the spectra were observed in the organic solvents.

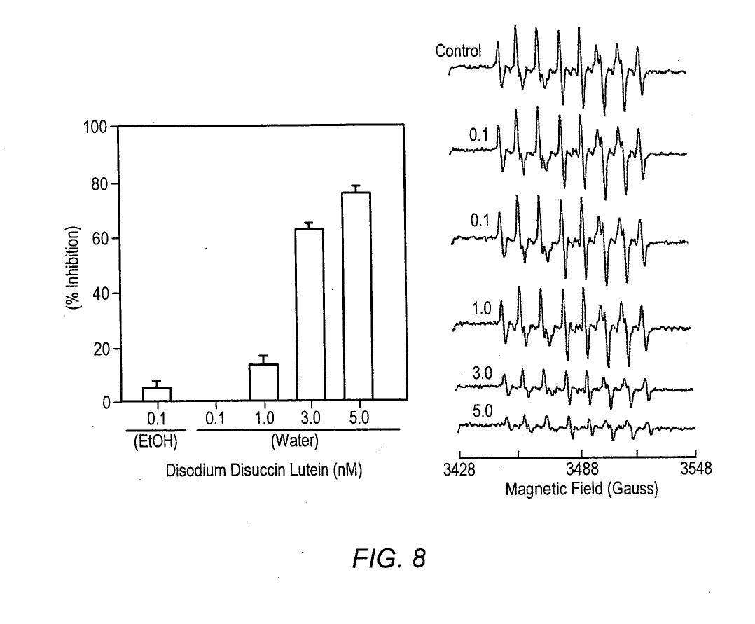

FIG. 7 depicts a UV/Vis absorption spectra of the disodium diphosphate derivative of natural source lutein in water (λₘₐₓ=428 nm) with increasing concentrations of ethanol. Concentration of the derivative was held constant for each increased concentration of EtOH in solution. The λₘₐₓ increases to 448 nm at an EtOH concentration of 40%, at which no further shift of the absorption maximum occurs (i.e. a molecular solution is reached).

Direct Superoxide Anion Scavenging by EPR Spectroscopy

The mean percent inhibition of superoxide anion signal (±SEM) as detected by DEPMPO spin-trap by the disodium disuccinate derivative of natural source lutein (tested in water) is shown in FIG. 8. A 100 μM formulation (0.1 mM) was also tested in 40% EtOH, a concentration shown to produce a molecular (i.e. non-aggregated) solution. As the concentration of the derivative increased, inhibition of superoxide anion signal increased in a dose-dependent manner. At 5 mM, approximately 34 (75%) of the superoxide anion signal was inhibited. No significant scavenging (0% inhibition) was observed at 0.1 mM in water. Addition of 40% EtOH to the derivative solution at 0.1 mM did not significantly increase scavenging over that provided by the EtOH vehicle alone (5% inhibition). The millimolar concentration scavenging by the derivative was accomplished in water alone, without the addition of organic co-solvent (e.g., acetone, EtOH), heat, detergents, or other additives. This data suggested that card-pack aggregation for this derivative was not occurring in aqueous solution (and thus limiting the interaction of the aggregated carotenoid derivative with aqueous superoxide anion).

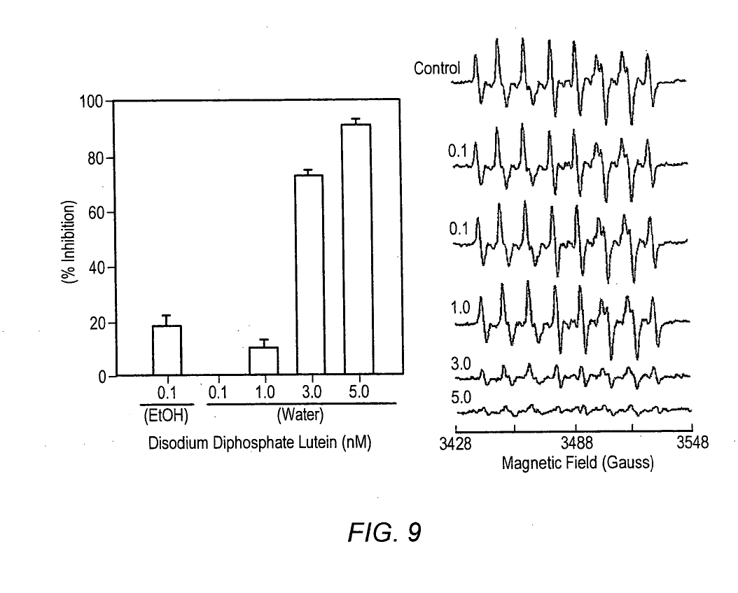

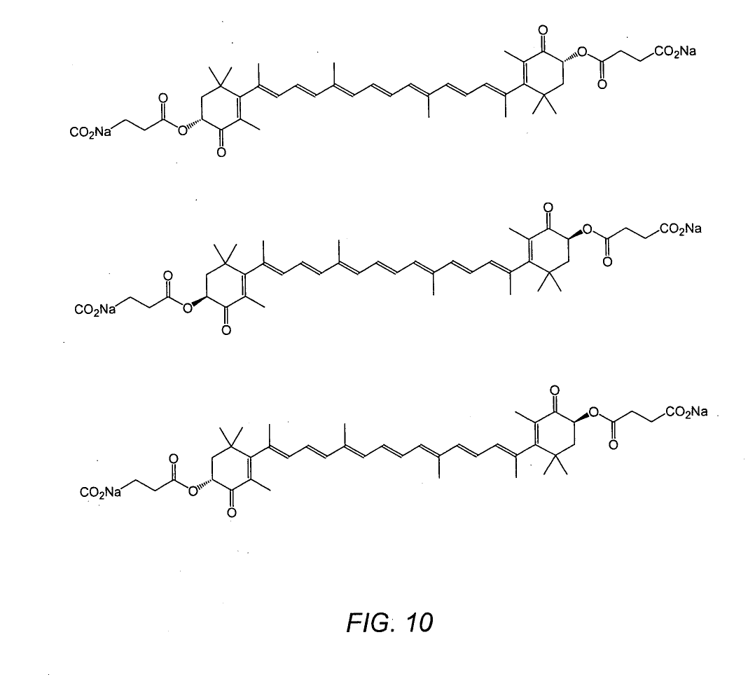

The mean percent inhibition of superoxide anion signal (±SEM) as detected by DEPMPO spin-trap by the disodium diphosphate derivative of natural source lutein (tested in water) is shown in FIG. 9. A 100 μM formulation (0.1 mM) was also tested in 40% EtOH, a concentration also shown to produce a molecular (i.e. non-aggregated) solution of this derivative. As the concentration of the derivative increased, inhibition of the superoxide anion signal increased in a dose-dependent manner. At 5 mM, slightly more than 90% of the superoxide anion signal was inhibited (versus 75% for the disuccinate lutein sodium salt). As for the disuccinate lutein sodium salt, no apparent scavenging (0% inhibition) was observed at 0.1 mM in water. However, a significant increase over background scavenging by the EtOH vehicle (5%) was observed after the addition of 40% EtOH, resulting in a mean 18% inhibition of superoxide anion signal. This suggested that disaggregation of the compound lead to an increase in scavenging ability by this derivative, pointing to slightly increased scavenging ability of molecular solutions of the more water-dispersible diphosphate derivative relative to the disuccinate derivative. Again, the millimolar concentration scavenging by the derivative was accomplished in water alone, without the addition of organic co-solvent (e.g., acetone, EtOH), heat, detergents, or other additives.

TABLE I
Descriptive statistics of mean % inhibition of superoxide anion signal for aqueous and ethanolic
(40%) formulations of disodium disuccinate derivatives of natural source lutein tested in
the current study. Sample sizes of 3 were evaluated for each formulation, with the exception
of natural source lutein in 40% EtOH stock solution (N = 1). Mean % inhibition did not
increase over background levels until sample concentration reached 1 mM in water; likewise,
addition of 40% EtOH at the 0.1 mM concentration did not increase scavenging over background
levels attributable to the EtOH vehicle (mean = 5% inhibition).

| | | | | Mean (% | | | | | |
|---|---|---|---|---|---|---|---|---|---|
| Sample | Solvent | Concentration | N | inhibition) | S.D. | SEM | Min | Max | Range |
| Lutein Disuccinate | 40% | 0.1 mM | 3 | 5.0 | 4.4 | 2.5 | 0 | 8 | 8 |
| Sodium Salt | EtOH | | | | | | | | |
| Lutein Disuccinate | Water | 0.1 mM | 1 | 0.0 | ND | ND | 0 | 0 | 0 |
| Sodium Salt | | | | | | | | | |
| Lutein Disuccinate | Water | 1.0 mM | 3 | 13.0 | 5.6 | 3.2 | 8 | 19 | 11 |
| Sodium Salt | | | | | | | | | |
| Lutein Disuccinate | Water | 3.0 mM | 3 | 61.7 | 4.0 | 2.3 | 58 | 66 | 8 |
| Sodium Salt | | | | | | | | | |
| Lutein Disuccinate | Water | 5.0 mM | 3 | 74.7 | 4.5 | 2.6 | 70 | 79 | 9 |
| Sodium Salt | | | | | | | | | |

TABLE II
Descriptive statistics of mean % inhibition of superoxide anion signal for aqueous and ethanolic
(40%) formulations of disodium diphosphate derivatives of natural source lutein tested in the current
study. Sample sizes of 3 were evaluated for each formulation, with the exception of lutein diphosphate
in water at 100 μM (0.1 mM) where N = 1. Mean % inhibition of superoxide anion signal increased
in a dose-dependent manner as the concentration of lutein diphosphate was increased in the test
assay. At 100 μM in water, no inhibition of scavenging was seen. The molecular solution in 40%
EtOH (mean % inhibition = 18%) was increased above background scavenging (5%) by the ethanolic
vehicle, suggesting that disaggregation increased scavenging at that concentration. Slightly increased
scavenging (on a molar basis) may have been obtained with the diphosphate derivative in comparison
to disuccinate derivative (see Table 1 and FIG. 8).

| | | | | Mean (% | | | | | |
|---|---|---|---|---|---|---|---|---|---|
| Sample | Solvent | Concentration | N | inhibition) | S.D. | SEM | Min | Max | Range |
| Lutein Diphosphate. | 40% | 0.1 mM | 3 | 18.0 | 7.0 | 4.0 | 11 | 25 | 14 |
| Sodium Salt | EtOH | | | | | | | | |
| Lutein Diphosphate | Water | 0.1 mM | 1 | 0.0 | ND | ND | 0 | 0 | 0 |
| Sodium Salt | | | | | | | | | |
| Lutein Diphosphate | Water | 1.0 mM | 3 | 9.3 | 3.5 | 2.0 | 6 | 13 | 7 |
| Sodium Salt | | | | | | | | | |
| Lutein Diphosphate | Water | 3.0 mM | 3 | 72.3 | 3.1 | 1.8 | 69 | 75 | 6 |
| Sodium Salt | | | | | | | | | |
| Lutein Diphosphate | Water | 5.0 mM | 3 | 91.0 | 2.6 | 1.5 | 88 | 93 | 5 |
| Sodium Salt | | | | | | | | | |

In the current study, facile preparations of the disodium disuccinate and tetrasodium phosphate esters of natural source (RRR) lutein are described. These asymmetric C40 carotenoid derivatives exhibited aqueous dispersibility of 2.85 and 29.27 mg/mL, respectively. Evidence for both card-pack (H-type) and head-to-tail (J-type) supramolecular assembly was obtained with UV-V is spectroscopy for the aqueous solutions of these compounds. Electronic paramagnetic spectroscopy of direct aqueous superoxide scavenging by these derivatives demonstrated nearly identical dose-dependent scavenging profiles, with slightly increased scavenging noted for the diphosphate derivative. In each case, scavenging in the millimolar range was observed. These results show that as parenteral soft drugs with aqueous radical scavenging activity, both compounds are useful in those clinical applications in which rapid and/or intravenous delivery is desired for the desired therapeutic effect(s).

Overview of Methods Used

The measurement of antioxidant activity is highly dependent on the experimental system used, and one should be cautious in comparing results of separate studies, or of extending the conclusions of a given study beyond its experimental limits.

Proton electron double resonance imaging (PEDRI) is a recently developed low-field double magnetic resonance technique (Lurie et al. 2002). The technique uses the Overhauser effect to image the distribution of free radicals in biological samples. Of particular interest is that the technique can be extended to whole animals, allowing systemic redox state analysis as well as individual target organ analysis after therapeutic treatment. The in vivo application of the technique requires a balance between the electron paramagnetic resonance (EPR) irradiation frequency and the magnetic field applied: sufficiently low magnetic field must be utilized that allows penetration of the whole animal, while at the same time limiting nonresonant power deposition in the animal. Conversion of a commercially available whole-body MRI system for operation as a small-animal PEDRI imager has been described previously, and adapted for use in the examples described herein (Lurie et al. 2002).

PEDRI has certain advantages, such as high spatial and temporal resolution, over the traditional CW EPR imaging technique. The prior art extensively describe the use of PEDRI to image the distribution and pharmacokinetics of a paramagnetic spin probe (a triaryl methyl radical, TAM) in a murine model (Li et al. 2002). PEDRI allowed rapid imaging of the distribution and clearance of this paramagnetic probe, suggesting potential preclinical utility in the analysis of oximetry and redox state analysis in mice. PEDRI has also been applied to image nitroxide free radicals in isolated beating rat hearts (Liebgott et al. 2003). The change in radical distribution within the heart during infusion and clearance of the TEMPONE spin probe may be readily measured using techniques described in the prior art.

The nitroxide spin probes undergo reduction to an EPR-silent hydroxylamine form after parenteral infusion. The rate of reduction of nitroxide in living biological tissues is an indirect measure of oxidant stress (Liebgott et al. 2003). The greater the systemic (and/or target organ) oxidant stress, the more rapid is the rate of reduction of the concentration of the infused spin probe, and hence the loss of measured PEDRI signal. Therefore, by measuring the distribution, clearance and metabolism of the nitroxide spin probe using PEDRI, the redox status of whole animals can be established. Although the technique does not provide absolute measurements of redox status, relative changes can be readily established by comparing the data from treated animals with matched controls.

### EXPERIMENTAL PROCEDURES

Materials

Cardax™ was synthesized from crystalline astaxanthin [3R,3′R, 3R,3′S (meso), and 3S,3′S (1:2:1)], a mixture of stereoisomers obtained commercially (Buckton Scott, India), as previously described (Frey et al. 2004). The all-trans (all-E) form of the mixture of stereoisomers used was a linear, rigid molecule (“bolaamphiphile”) owing to the lack of cis (or Z) configuration(s) in the polyene chain of the spacer material (Foss et al. 2005). The disodium disuccinate derivative of synthetic astaxanthin was successfully synthesized at >97% purity by HPLC (as AUC; chemical structures of individual stereoisomers shown in FIG. 1).

### Example 1

Animal Dosing and PEDRI Measurements

Individual mice (average body weight ˜30 g) were treated with either Cardax™ (10 mg/kg) or vehicle (normal saline) delivered via daily I.V. injection in the jugular vein for a period of 4 days. Following the last injection, the animals were anesthetized using ketamine/xylazine, and ˜0.6 mL of the paramagnetic spin probe TEMPONE (100 mM in PBS) was infused via tail vein. TEMPONE (4-oxo-2,2,6,6,-tetramethylpiperidinyl-N-oxy) is a commonly used redox probe, and is suitable for use in PEDRI (Liebgott et al. 2003). TEMPONE exhibits a high metabolic clearance rate, and a relatively narrow EPR line width. The PEDRI images were acquired immediately following infusion of the redox probe.

After the mice were placed in the PEDRI resonator, a bolus injection of 0.55-0.6 mL (normalized to the body weight of the individual animal) of 100 mM TEMPONE solution in PBS was given over 50-55 s through the tail vein. A series of time-course PEDRI measurements were then acquired every 10-15 s after the injection of the nitroxide probe solution, until the image intensity reached a minimum (i.e., no nitroxide signal could be detected). In order to follow the rapid oxidation process, a custom 2D PEDRI sequence analysis (based on standard fast spin echo pulse sequence) was used. The following instrument parameters were utilized: NMR Frequency, 847 kHz; EPR Frequency, 561 MHz; Receiver band-width, 10 kHz; Slice thickness, 25 mm; Field of volume (FOV), 8 cm (cropped to 6 cm in the final image); Image matrix, 64×64 pixels; EPR irradiation power, ˜2 W; EPR irradiation time, 4×400 ms×2=3.2 s; Time of acqusition, 2×(430 ms+400 ms)×4≈7 s.

### Example 2

Image Intensity Fitting for the Region of Interest (Heart)

The log normal equation below was utilized to fit the image intensity in the region of interest in each mouse (myocardium):

$$Equation 1 \text{:}$$

$$y = {y}_{0} + a {ⅇ}^{〈 - {0.5 [ \frac{ln ( \frac{x}{{x}_{0}} )}{b} ]}^{2} 〉}$$

The “b” values represent the rate of decay of the paramagnetic spin probe after infusion into the tail vein of each animal. As b increases, the rate of decay of the image intensity signal decreases from peak values obtained in the region of interest.

### Example 3

Statistical Analysis

Statistical analyses were performed with the Prism 4 package (GraphPad Software Inc., San Diego, Calif.). Student's t-test on the mean of the treatment group versus the control group was performed. Data are reported as mean±standard error of the mean (mean±SEM). * denotes significance at the P<0.05 level; ** denotes significance at the P<0.01 level.

### Example 4

Results

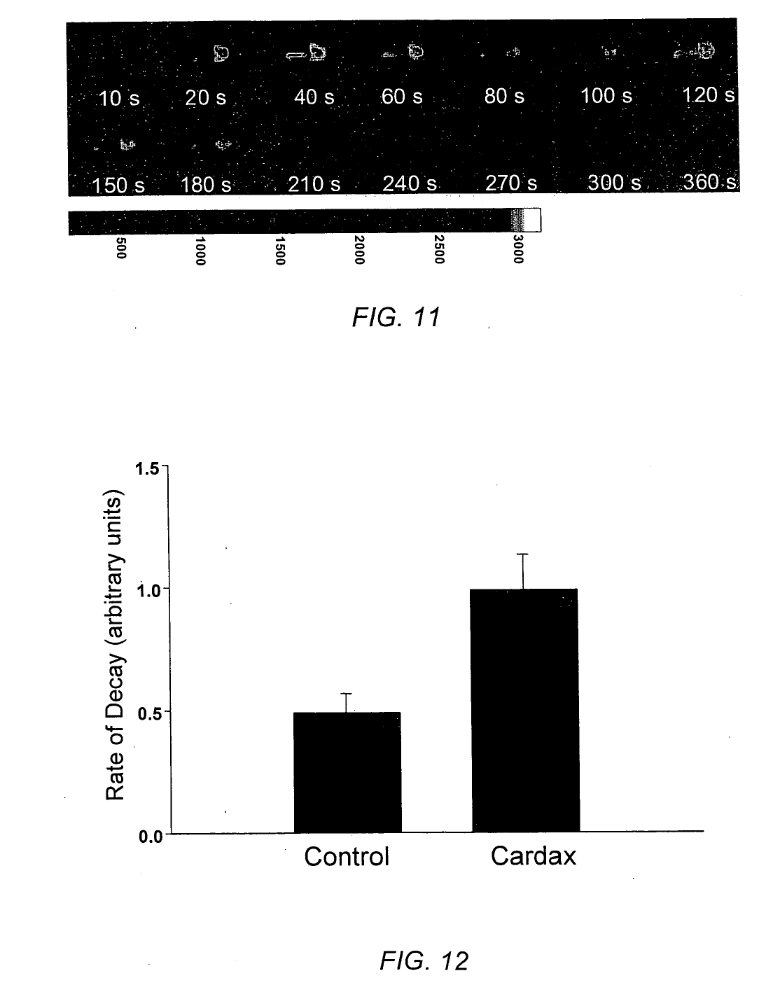

Four untreated (control) mice and four mice treated with Cardax™ as indicated were used in these studies. Whole-body time course PEDRI analysis was performed on each mouse (see FIG. 11 for a representative series). Image analysis was subsequently performed in the myocardial region for each mouse. Image data from the myocardial region were fitted using a single exponential pharmacokinetic function to obtain half-time (t) for the decay of the nitroxide signal intensity. The half-time is inversely proportional to the decay rate; thus a larger value of t corresponds to a slower rate of decay of the nitroxide probe. A significant difference in the mean t values between the control and treated mice was observed (FIG. 12). Treated mice exhibited a significantly higher mean t value (0.99±0.14 min) compared to the untreated mice (0.49±0.08). Therefore, Cardax™-treated mice showed a slower mean rate of nitroxide reduction in the mouse heart as compared to the untreated control animals.

### CONCLUSION

The rate of decay of the paramagnetic spin probe TEMPONE is dependent on both the cellular redox environment of the tissue being examined, as well as systemic redox status. TEMPONE is susceptible to both bioreduction in solid organ tissue as well as direct oxidation. Therefore, a decrease in circulating and/or cellular oxidizing species (e.g., ROS and other oxidant species) would be expected to reduce the decay of the infused spin probe. The results presented herein and discussed above therefore demonstrate that systemic administration of pharmaceutical compositions having the subject carotenoid analogs or derivatives (i) significantly alters the redox status of certain tissues (e.g., heart) and (ii) significantly reduces the oxidative stress in the heart of subject animals who received the pharmaceutical preparations.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

### ADDITIONAL REFERENCES

The following references, to the extent that they provide exemplary procedural or other details supplementary to those set forth herein, are specifically incorporated herein by reference.
Bangham A D, Standish M M, Watkins J C. Diffusion of univalent ions across the lamellae of swollen phospholipids. J. Mol. Biol. 1965; 13:238-252.
Bolli R, Jeroudi M O, Patel B S, Aruoma O I, Halliwell B, Lai E K and McCay P B (1989) Marked reduction of free radical generation and contractile dysfunction by antioxidant therapy begun at the time of reperfusion. Evidence that myocardial “stunning” is a manifestation of reperfusion injury. Circ Res 65:607-622.
Britton G (1995) Structure and properties of carotenoids in relation to function. Faseb J 9:1551-1558.
Cantrell A, McGarvey D J, Truscott T G, Rancan F and Bohm F (2003) Singlet oxygen quenching by dietary carotenoids in a model membrane environment. Arch Biochem Biophys 412:47-54.
Cardounel A J, Dumitrescu C, Zweier J L and Lockwood S F (2003) Direct superoxide anion scavenging by a disodium disuccinate astaxanthin derivative: Relative efficacy of individual stereoisomers versus the statistical mixture of stereoisomers by electron paramagnetic resonance imaging. Biochem Biophys Res Commun 307:704-712.
Chung B H, Segrest J P, Ray M J, Brunzell J D, Hokanson J E, Krauss R M, Beaudrie K, Cone J T. Single vertical spin density gradient ultracentrifugation. Methods Enzymol. 1986; 128:181-208.
de Córdoba et al., “The Human Complement Factor H: Functional Roles, Genetic Variations and Disease Associations” Molecular Immunology Vol. 41, 2004, pp. 355-367
Curry S, Brick P and Franks N P (1999) Fatty acid binding to human serum albumin: new insights from crystallographic studies. Biochim Biophys Acta 1441:131-140.
de Beer F C, Hind C R, Fox K M, Allan R M, Maseri A and Pepys M B (1982) Measurement of serum C-reactive protein concentration in myocardial ischaemia and infarction. Br Heart J 47:239-243.
Devasagayam T P, Werner T, Ippendorf H, Martin H D and Sies H (1992) Synthetic carotenoids, novel polyene polyketones and new capsorubin isomers as efficient quenchers of singlet molecular oxygen. Photochem Photobiol 55:511-514.
Diaz Padilla N, Bleeker W K, Lubbers Y, Rigter G M, Van Mierlo G J, Daha M R and Hack C E (2003) Rat C-reactive protein activates the autologous complement system. Immunology 109:564-571.
Edwards et al., “Complement Factor H Polymorphism and Age-Related Macular Degeneration” published online 10 Mar. 2005; 10.1126/science.1110189
El-Saadani, M., Esterbauer, H., El-Sayed, M., Goher, M., Nassar, A. Y., and Jurgens, G. (1989) A spectrophotometric assay for lipid peroxides in serum lipoproteins using a commercially available reagent. J. Lipid Research. 30(4), 627-630.
Esterbauer H, Striegl G, Puhl H, Rotheneder M. Continuous monitoring of in vitro oxidation of human low density lipoprotein. Free Radic. Res. Commun. 1989; 6:67-75.
Funkhouser T A and Vik D P (1999) Complement receptor type 1 gene regulation: retinoic acid and cytosine arabinoside increase CR1 expression. Scand J Immunol 49:21-28.
Gross G J and Lockwood S (In Press) Acute and chronic administration of disodium disuccinate astaxanthin (Cardax™) produces marked cardioprotection in dog hearts. Molecular and Cellular Biochemistry.
Gross G J and Lockwood S F (2004) Cardioprotection and myocardial salvage by a disodium disuccinate astaxanthin derivative (Cardax). Life Sci 75:215-224.
Gruszecki, W. I. (1999) in The photochemistry of carotenoids. Frank, H. A., Young, A. J., Britton, G., and Cogdell, R. J., Eds. pp. 363-379, Kluwer Academic, Dordrecht, The Netherlands.
Haines et al., “Complement Factor H Variant Increases the Risk of Age-Related Macular Degeneration” published online 10 Mar. 2005; 10.1126/science.1110359
Halliwell, B. (1997) Antioxidants and human disease: A general introduction. Nutr. Rev. 55, 544-552.
Huang D, Ou B, Hampsch-Woodill M, Flanagan J A, Prior R L. High-throughput assay of oxygen radical absorbance capacity (ORAC) using a multichannel liquid handling system coupled with a microplate fluorescence reader in 96-well format. J Agric Food Chem. 2002; 50:1815-1821.
Jensen S K, Engberg R M and Hedemann M S (1999) All-rac-alpha-tocopherol acetate is a better vitamin E source than all-rac-alpha-tocopherol succinate for broilers. J Nutr 129:1355-1360.
Jolly S R, Kane W J, Bailie M B, Abrams G D and Lucchesi B R (1984) Canine myocardial reperfusion injury. Its reduction by the combined administration of superoxide dismutase and catalase. Circ Res 54:277-285.
Kilgore K S, Friedrichs G S, Johnson C R, Schasteen C S, Riley D P, Weiss R H, Ryan U and Lucchesi B R (1994) Protective effects of the SOD-mimetic SC-52608 against ischemia/reperfusion damage in the rabbit isolated heart. J Mol Cell Cardiol 26:995-1006
Klein et al., “Complement Factor H Polymorphism in Age-Related Macular Degeneration” published online 10 Mar. 2005; 10.1126/science.1109557
Laemmli U K. Cleavage of structural proteins during the assembly of the head of bacteriophage. Nature. 1970; 227:680-686.
Lauver D A, Booth E A, White A J, Poradosu E and Lucchesi B R (2005) Sulodexide Attenuates Myocardial Ischemia/Reperfusion Injury and the Deposition of C-Reactive Protein in Areas of Infarction without Affecting Hemostasis. J Pharmacol Exp Ther 312:794-800.
Lawlor, S. M., and O'Brien, N. M. (1995) Astaxanthin: antioxidant effects in chicken embryo fibroblasts. Nutr. Res. 15, 1695-1704.
Lucchesi B R (1994) Complement, neutrophils and free radicals: mediators of reperfusion injury. Arzneimittelforschung 44:420-432.
Mathews-Roth, M. (1991) Recent progress in the medical applications of carotenoids. Pure Appl. Chem. 63, 147-156.
Mayne, S. T. (1996) Beta-carotene, carotenoids and disease prevention in humans. FASEB J. 10, 690-701
Mercadante, A. (1999) New carotenoids: recent progress. 12^{th} International Carotenoid Symposium, Cairns, Australia, July 1999.
Miki, W. (1991) Biological functions and activities of animal carotenoids. Pure Appl. Chem. 63, 141-146
Nijmeijer R, Lagrand W K, Lubbers Y T, Visser C A, Meijer C J, Niessen H W and Hack C E (2003) C-reactive protein activates complement in infarcted human myocardium. Am J Pathol 163:269-275.
Ong, A. S. H., and Tee. E. S. (1992) Natural sources of carotenoids from plants and oils. Meth. Enzymol. 213, 142-167.
Osterlie M, Bjerkeng B and Liaaen-Jensen S (2000) Plasma appearance and distribution of astaxanthin E/Z and R/S isomers in plasma lipoproteins of men after single dose administration of astaxanthin(1). J Nutr Biochem 11:482-490.
Palozza, P., and Krinsky, N. I. (1992) Astaxanthin and canthaxanthin are potent antioxidants in a membrane model. Arch. Biochem. Biophys. 297, 291-295.
Park J. L. and Lucchesi B R (1999) Mechanisms of myocardial reperfusion injury. Ann Thorac Surg 68:1905-1912.
Pascual M, Catana E, Spertini F, Macon K, Volanakis J E and Schifferli J A (1990) A monoclonal antibody which blocks the function of factor D of human complement. J Immunol Methods 127:263-269.
Peto, R., Doll, R., Buckley, J. D., and Spom, M. B. (1981) Nature 290, 201-208.
Ridker et al. “C-Reactive Protein Levels and Outcomes after Statin Therapy” The New England Journal of Medicine, 2005, 352, pp 20-28.
Showalter L A, Weinman S A, Osterlie M and Lockwood S F (2004) Plasma appearance and tissue accumulation of non-esterified, free astaxanthin in C57BL/6 mice after oral dosing of a disodium disuccinate diester of astaxanthin (Heptax). Comp Biochem Physiol C Toxicol Pharmacol 137:227-236.
Simonyi M, Bikadi Z, Zsila F and Deli J (2003) Supramolecular exciton chirality of carotenoid aggregates. Chirality 15:680-698.
Subczynski, W. K., Markowska, E., and Sielewiesiuk, J. (1991) Effect of polar carotenoids on the oxygen diffusion-concentration product in lipid bilayers. An EPR spin label study. Biochim Biophys Acta 1068, 68-72.
Tanaka, T., Makita, H., Ohnishi, M., Mori, H., Satoh, K., and Hara, A. Chemoprevention of rat oral carcinogenesis by naturally occurring xanthophylls, astaxanthin and canthaxanthin. (1995) Cancer Res. 55, 4059-4064.
Tanhehco E J, Yasojima K, McGeer P L, Washington R A and Lucchesi B R (2000) Free radicals upregulate complement expression in rabbit isolated heart. Am J Physiol Heart Circ Physiol 279:H195-201.
Terao, J. (1989) Antioxidant activity of beta-carotene-related carotenoids in solution. Lipids 24, 659-661.
Venugopal et al. “Macrophage Conditioned Medium Induces the Expression of C-Reactive Protein in Human Aortic Endothelial Cells: Potential for Paracrine/Autocrine Effects” American Journal of Pathology, 2005, 166(4), pp 1265-1271.
Volanakis J E (1982) Complement activation by C-reactive protein complexes. Ann N Y Acad Sci 389:235-250.
Walter M F, Blumberg J B, Dolnikowski G G, Handelman G J. Streamlined F2-isoprostane analysis in plasma and urine with high-performance liquid chromatography and gas chromatography/mass spectroscopy. Anal. Biochem. 2000; 280:73-79.
Walter et al. “Sulfone COX-2 inhibitors increase susceptibility of human LDL and plasma to oxidative modification: Comparison to sulfonamide COX-2 inhibitors and NSAIDs”, Atherosclerosis 1 Vol. 77 (2004), pp. 235-243
Whaley K (1985) Methods in Complement for Clinical Immunologists. Churchill Livingstone, New York.
Yasojima K, Schwab C, McGeer E G and McGeer P L (1998) Human heart generates complement proteins that are upregulated and activated after myocardial infarction. Circ Res 83:860-869.
Yeh E T, Anderson H V, Pasceri V and Willerson J T (2001) C-reactive protein: linking inflammation to cardiovascular complications. Circulation 104:974-975.
Ytrehus K, Myklebust R, Olsen R and Mjos O D (1987) Ultrastructural changes induced in the isolated rat heart by enzymatically generated oxygen radicals. J Mol Cell Cardiol 19:379-389.
Ziegler, R. G., Mayne, S. T., and Swanson, C. A. (1996) Nutrition and lung cancer. Cancer Causes Control 7, 157-177.
Zsila F, Simonyi M and Lockwood S F (2003) Interaction of the disodium disuccinate derivative of meso-astaxanthin with human serum albumin: from chiral complexation to self-assembly. Bioorg Med Chem Lett 13:4093-4100.

1. A method of reducing cardiac oxidative stress in a human subject comprising administering to a human subject in need thereof a therapeutically effective amount of a pharmaceutically acceptable composition comprising one or more carotenoids, carotenoid analogs, or carotenoid derivatives having the structure: 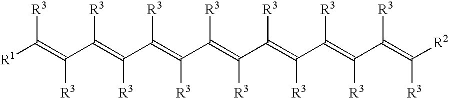
where each R³ is independently hydrogen or methyl, and where each R¹ and R² are independently: 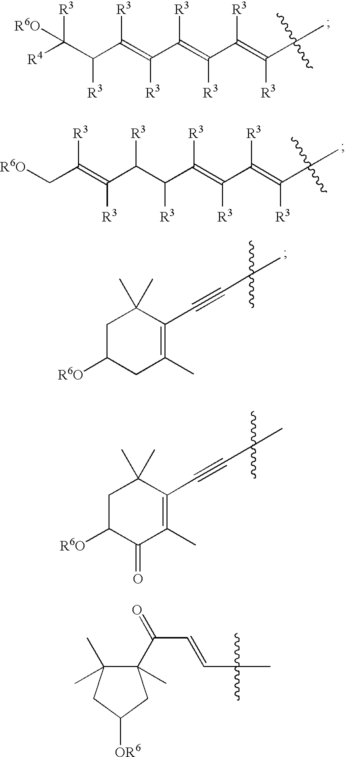
where R⁴ is hydrogen or methyl; wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

2. (canceled)

3. The method of claim 1, wherein the composition comprises one or more carotenoid derivatives or analogues having the structure: 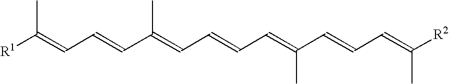
where each R³ is independently hydrogen or methyl, and where each R¹ and R² are independently: 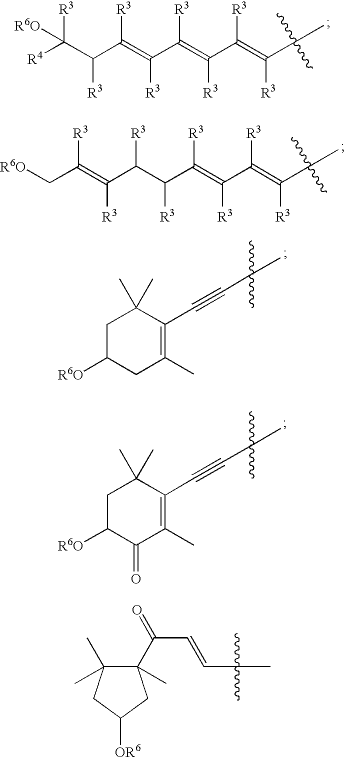
where R⁴ is hydrogen or methyl; wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

4. The method of claim 1, wherein the substituent —OR⁶ comprises: 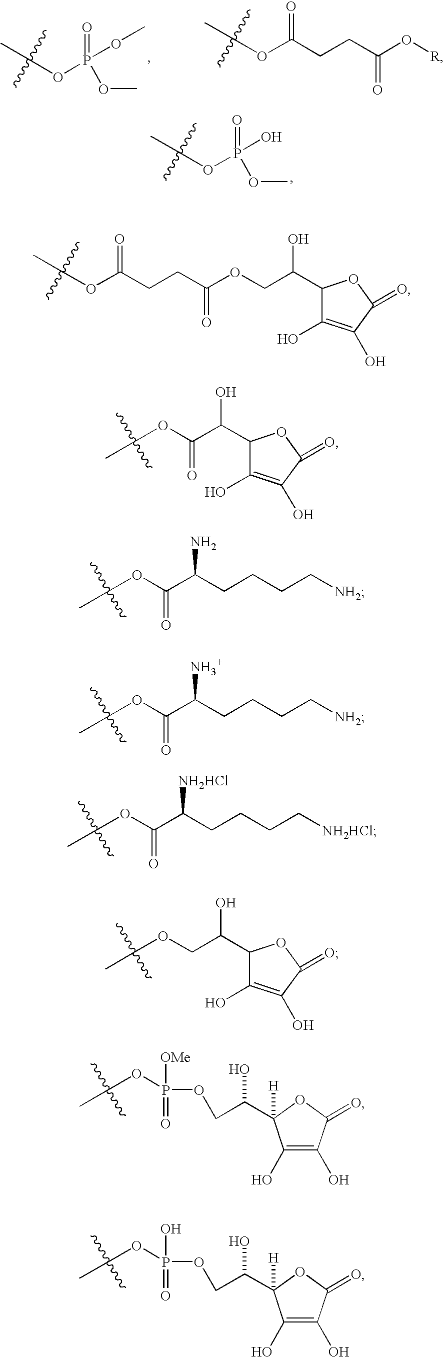 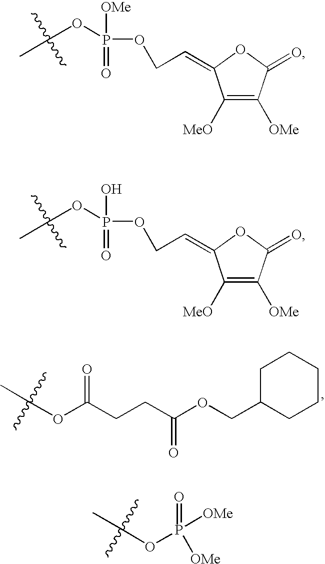
; and wherein each R is independently H, alkyl, aryl, benzyl, Group IA metal, or co-antioxidant.

5. The method of claim 1, wherein the substituent R⁶ in at least a portion of the carotenoid analogs or derivatives administered to the human subject is cleaved during use, and wherein one or more cleavage products are biologically active.

6. The method of claim 1, wherein the substituent —OR⁶ comprises 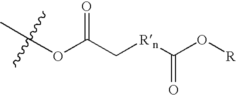
; where each R′ is independently —(CH₂)ₙ—, where n=1-10; and where each R is independently H, alkyl, aryl, benzyl, Group IA metal, or a co-antioxidant.

7. The method of claim 1, wherein the substituent —OR⁶ comprises 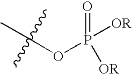
; where each R is independently H, alkyl, aryl, benzyl, Group IA metal, or a co-antioxidant.

8. The method of claim 1, wherein the substituent —OR⁶ comprises 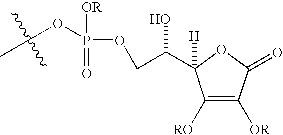
; where each R is independently H, alkyl, aryl, benzyl, or a Group IA metal.

9. The method of claim 1, wherein the composition comprises one or more carotenoid derivatives or analogues having the structure: 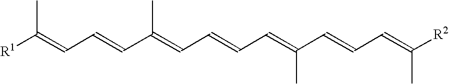
where each R³ is independently hydrogen or methyl, and where each R¹ and R² are independently: 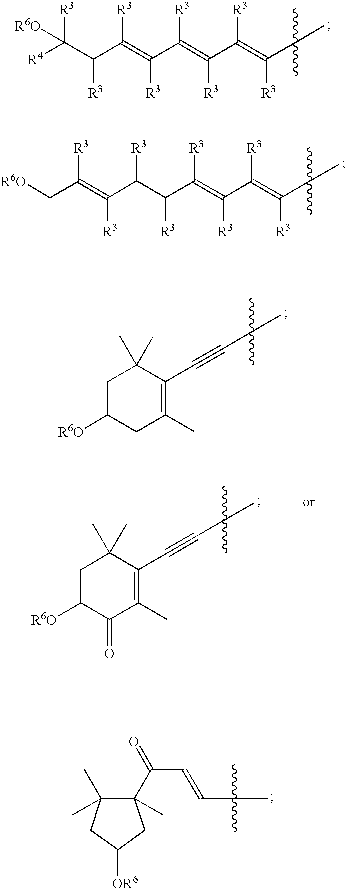
or
where R⁴ is hydrogen or methyl; and where R⁶ is

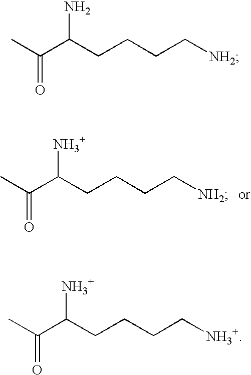

10. The method of claim 1, wherein the co-antioxidant comprises Vitamin C, Vitamin C analogs, Vitamin C derivatives, Vitamin E, Vitamin E analogs, Vitamin E derivatives, flavonoids, flavonoid derivatives, or flavonoid analogs.

11. The method of claim 10, wherein the flavonoids comprise quercetin, xanthohumol, isoxanthohumol, or genistein.

12. The method of claim 1, wherein the composition is administered orally.

13. The method of claim 1, wherein the composition is administered parenterally.

14. The method of claim 1, wherein the composition is administered as an aqueous solution.

15. The method of claim 1, wherein the composition is administered as an aqueous dispersion.

16. The method of claim 1, wherein the composition is administered intravenously.

17. The method of claim 1, wherein the composition is administered by intramuscular injection.

18-69. (canceled)

70. A pharmaceutical composition suitable for the treatment of a cardiac disorder associated with oxidative stress, said pharmaceutical composition comprising a therapeutically effective amount of one or more carotenoid analogs or derivatives in an amount sufficient to at least partially reduce oxidative stress in the heart of a subject administered said pharmaceutical composition, wherein one or more of the carotenoid analogs or derivatives have the structure: 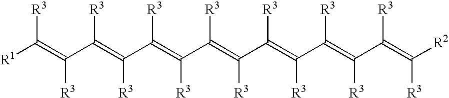
where each R³ is independently hydrogen or methyl, and where each R¹ and R² are independently: 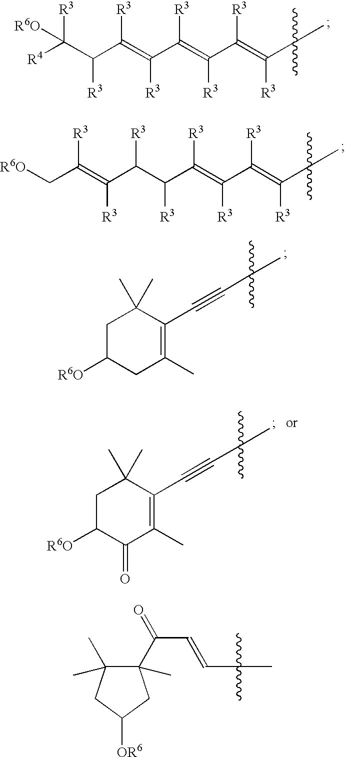
where R⁴ is hydrogen or methyl; wherein each R⁶ is independently: hydrogen; alkyl; aryl; -alkyl-N(R⁷)₂; -aryl-N(R⁷)₂; -alkyl-N⁺(R⁷)₃; -aryl-N⁺(R⁷)₃; -alkyl-CO₂H; -aryl-CO₂H; -alkyl-CO₂—, -aryl-CO₂—, —C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

71. The pharmaceutical composition of claim 70, wherein the substituent —OR⁶ comprises: 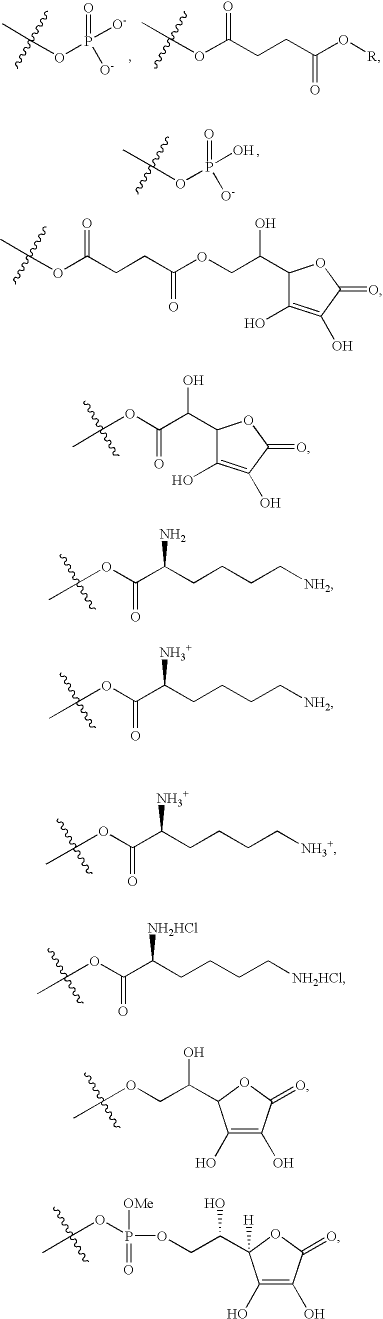 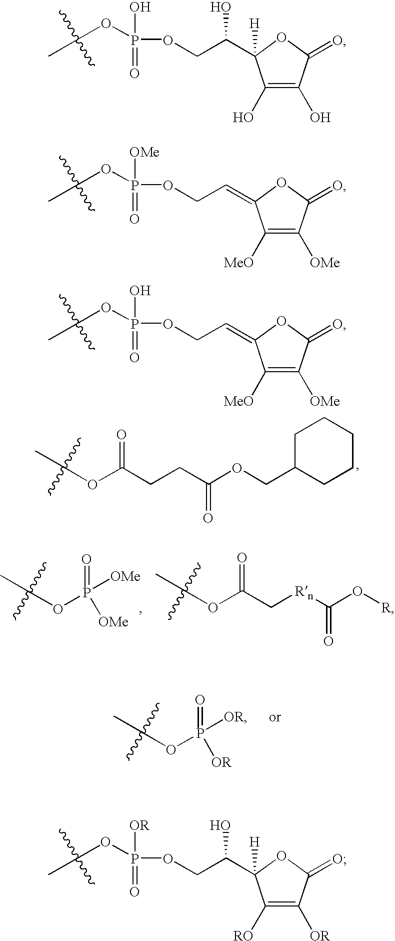
where each R′ is independently —(CH₂)ₙ—, where n=1-10; and where each R is independently H, alkyl, aryl, benzyl, Group IA metal, or a co-antioxidant.

72. The pharmaceutical composition of claim 70, wherein the co-antioxidant comprises Vitamin C, Vitamin C analogs, Vitamin C derivatives, Vitamin E, Vitamin E analogs, Vitamin E derivatives, flavonoids, flavonoid derivatives, or flavonoid analogs.

73. The pharmaceutical composition of claim 70, wherein the flavonoids comprise quercetin, xanthohumol, isoxanthohumol, or genistein.
